(19) United States
(12) Patent Application Publication
(10) Pub. No.: US 2004/0043483 A1
(43) Pub. Date: Mar. 4, 2004

(54) Novel tolerogenic dendritic cells and therapeutic uses therefor

(76) Inventors: Shiguang Qian, Allison Park, PA (US); Lina Lu, Allison Park, PA (US); John Fung, Pittsburgh, PA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/455,237

(22) Filed: Jun. 4, 2003

Related U.S. Application Data
(60) Provisional application No. 60/385,491, filed on Jun. 4, 2002.

(51) Int. Cl. A61K 48/00; C12N 5/08; A61K 31/66; A61K 31/522

(52) U.S. Cl. 435/372; 424/93.21; 514/109; 514/11; 514/171; 514/263.31; 514/291

(57) ABSTRACT

The present invention relates to tolerogenic dendritic cells (DCs) and methods for enriching for these cells in tissue preparations and using the cells for preventing or minimizing transplant rejection or for treating or preventing an autoimmune disease.

### RELATED APPLICATION INFORMATION

This application claims the benefit of U.S. Provisional Application No. 60/385,491, filed Jun. 4, 2002, the contents of which are specifically incorporated by reference herein.

### STATEMENT OF RIGHTS

This invention was supported in part by grant number DK 29661 from the National Institutes of Health. The U.S. Government may therefore have certain rights in the invention.

### FIELD OF THE INVENTION

The present invention features novel tolerogenic dendritic cells (DCs), which when administered to a subject, suppress the subject's immune response. The present invention further features methods for isolating tolerogenic DCs and for therapeutically using the cells for example to minimize transplant rejection or for treating a subject for an autoimmune disorder.

### BACKGROUND OF INVENTION

T cells and T Cell Differentiation

T lymphocytes, or T cells, function as the body's primary means of recognizing antigenic determinants on the cell surface. Interactions between specific ligands on the surface of the T cell and a cell displaying or presenting the antigen lead to T cell activation, which in turn leads to a variety of immunogenic responses, including cytokine synthesis and release, T and natural killer (NK) cell-mediated cytotoxicity, and activation of macrophages and B cells.

The course of T cell differentiation is crucial to the outcome of an immune response. Early in this process, T cells are committed to develop into one of several functionally distinct subsets, including Th1, Th2, and the recently described T regulatory (Tr) cells (Jonuleit et al., 2000, J. Exp. Med. 192:1213-1222). Tr cells are thought to play a critical role in the generation and maintenance of tolerance. Several varieties of Tr cells have been described, each with unique albeit somewhat nebulous characteristics. Thus, a number of definitions of Tr cells exist in the literature (Groux et al., 1997, Nature 389:737-742; Jonuleit et al., 2000, J. Exp. Med. 192:1213-1222; Roncarolo et al., 2001, J. Exp. Med. 193:5-9). Type 1 Tr (Tr1) cells are a subset characterized by their unique profile of cytokine production. Tr1 cells produce high levels of interleukin10 (IL-10), moderate amounts of transforming growth factor β (TGF-β) and IFN-γ but no interleukin-4 (IL-4) or interleukin-2 (IL-2). They exert immunoregulatory or suppressive effects (Groux et al., 1997. Nature 389:737-742; Saloga et al., 1999, Int. Arch. Allergy Immunol. 118:210-211; Jonuleit et al., 2000, J. Exp. Med. 192:1213-1222; Roncarolo et al., 2001, J. Exp. Med. 193:5-9).

T cell differentiation is regulated by the local microenvironment. Hence, the property of antigens (Ags) encountered by the T cell, and the expression of costimulatory molecules and cytokines by antigen presenting cells (APCs) strongly influence T cell differentiation. In vitro, interleukin12 (IL-12) promotes Th1 differentiation (Hsieh et al., 1993, Science 260:547-549; Schmitt et al., 1994, Eur. J. Immunol. 24:343-347), whereas IL-4 promotes Th2 differentiation (Swain et al., 1990, J. Immunol. 145:3796-3806; Kaplan et al., 1996, Immunity 4:313-319). Similarly, the generation of Tr1 cells is driven by IL-10 (Groux et al., 1997, Nature 389:737-742). The stimuli controlling T cell differentiation during an in vivo immune response are less clear.

Dendritic Cells and T Cell Differentiation and Activation

Dendritic cells are professional APCs uniquely suited for the activation of T cells (Steinman, 1991, Annu. Rev. Immunol. 9:271-296). Recent data suggest that different DC subsets provide T cells with selective signals that guide either Th1 or Th2 differentiation. In mice, DCs have been classified into myeloid and lymphoid subsets according to their phenotype and their development from distinct precursors (Ardavin et al., 1993, Nature 362:761-763; Wu et al., 1996, J. Exp. Med. 184:903-911; Vremec and Shortman, 1997, J. Immunol. 159:565-573; Steinman and Inaba, 1999, J Leukocyte Biol. 66:205-208; Steinman et al., 2000, J. Exp. Med. 191:411-416).

These subsets of DCs share a number of distinct properties, including dendritic morphology, the ability to migrate, and expression of a range of molecules required for activation of T cells. However, they differ in their regulation of the immune response. Thus, myeloid DCs usually initiate immune responses, and typically induce Th1 differentiation. In contrast, the so-called lymphoid DCs propagated in response to interleukin3 (IL-3), while capable of activating lymphocytes, may limit T cell proliferation by inducing Fas-mediated apoptosis and inhibiting cytokine production (Kronin et al., 1996, J. Immunol. 157:3819-3827; Suss and Shortman, 1996, J. Exp. Med. 183:1789-1796; Fazekas de St. Groth, 1998, Immunol. Today 19:448-454; Iwasaki and Kelsall, 1999, J. Exp. Med. 190:229-239).

Like mice, humans also contain two DC types developed from distinct precursors. DC1, propagated in response to granulocyte-macrophage colony stimulating factor (GM-CSF) from peripheral blood monocytes, produce high levels of IL-12 and induce Th1 differentiation, while DC2, propagated from blood or tonsil plasmacytoid T cells in response to IL-3, promote Th2 differentiation (Grouard et al., 1997, J. Exp. Med. 185:1101-1111; Rissoan et al. 1998; Science 283:1183-1186). Repetitive stimulation with allogeneic immature DCs induces IL-10-producing, nonproliferating T cells with regulatory properties (Jonuleit et al., 2000, J. Exp. Med. 192:1213-1222).

Tolerance and Autoimmune Disease

Autoimmune disorders are characterized by the loss of tolerance against self-antigens, activation of lymphocytes reactive against “self” antigens (autoantigens), and pathological damage in target organs. In most situations, autoimmunity may be prevented by peripheral tolerance, which is a process presumably involving a series of multi-step interactions between APCs, in particular DCs, and effector T cells.

DCs play a crucial role in controlling immune responses, and can either augment or reduce autoimmune responses by a variety of mechanisms. For example, in the autoimmune disease type 1 diabetes (T1D), antigens of the insulin-producing β-cells of the pancreas may be taken up and processed by DCs, leading to the generation of a cytotoxic T lymphocyte (CTL) response that destroys the remaining pancreatic 13 cells. Loss of B cell mass in turn prevents the secretion of insulin in response to changes in blood glucose levels, resulting in hyperglycemia and its associated pathologies.

This cytotoxic response is predominantly Th1-mediated, and the dose and timing of antigen delivery by DCs, the types of molecules expressed on the surfaces of DCs, and the pattern of cytokine production in these cells are the main parameters regulating the outcome of the autoimmune response in TID. For example, expression of IL-12, a cytokine released by activated DCs that induces a Th1 response, correlates with β-cell destruction in mouse models of diabetes (e.g. the non-obese diabetic or NOD mouse). Furthermore, the development of autoimmune diabetes in a mouse model is closely regulated by the interaction of the CD28 antigen on the surface of the T cell with the B7 antigen on the surface of the APC. Interaction of CD28 on T cells with CD86 (B7-2), a potent co-stimulatory molecule found on ‘mature’ DCs, is required for development of the disease. Although it is clear that DCs are involved in the presentation of MHC Class II restricted autoantigens during the development of autoimmune diabetes in the NOD mouse, the role of DCs in the regulation of the autoimmune state has not been delineated. The detailed understanding of the phenotype and function of DCs, particularly in the scenario of autoimmune predisposition, will lead to the development of therapeutic strategies to induce tolerance to self antigens, thereby preventing the development of T1D.

Tolerogenic DCs

A role for DCs in central tolerance induction was initially demonstrated in the context of self-tolerance within the thymus, in which DCs stimulate the deletion self-reactive T cells. Both myeloid and lymphoid DC populations have been reported to be able to induce peripheral, antigen-specific unresponsiveness in various experimental models, or have been implicated as having a role in self-tolerance. Mechanisms whereby DCs accomplish this goal include selective activation of Th2 subsets, induction of regulatory T cells, induction of T cell anergy, and induction of T cell apoptosis. The acceptance of this concept is facilitated by the identification of DC subsets, whose functions are affected (and perhaps dictated) by micro-environmental factors, in particular cytokines, IL-10, TGF-β, prostaglandin E2, and corticosteroids.

Other molecules also influence DC function. For example, the chimeric fusion protein cytotoxic T lymphocyte antigen 4 (CTLA4)-Ig can render DCs tolerogenic. Fas ligand (CD95 L) that is expressed on lymphoid or myeloid DCs and tumor necrosis factor-related apoptosis-inducing ligand (TRAIL) that is expressed on human CD11c⁺ blood DCs may regulate or eliminate T cells responding to antigens presented by DCs. Thus, genetically engineered DCs expressing immunomodulatory molecules, such as viral IL-10 (vIL-10), TGF-β, Fas ligand, or cytotoxic T lymphocyte antigen 4-immunoglobulin (CTLA4Ig) have been developed. For instance delivery of IL-10into mature DCs has been found to promote tolerogenicity. (Lu et al., 1999, J Leukoc. Biol. 66:293-296) and delivery of cytotoxic CTLA4Ig into mature DCs has also been shown to promote tolerogenicity and survival of these DCs in allogeneic recipients (Lu et al., 1999, Gene Ther. 6:554-563). In addition, delivery of TGF-β into DCs has been found to prevent the reduction of DCs generally seen with adenovirus infection and also increase the numbers and prolong the survival of the infected DCs in the spleen of a host to whom the DCs have been administered (Lee et al., 1998, Transplantation 66:1810-1817).

Methods of either propagating immature and undifferentiated mammalian DCs in the presence of a cytokines and/or extracellular matrix proteins or manipulating DCs to express various cytokines has been described, as has use of propagated or manipulated DCs to enhance tolerogenicity (See for example, U.S. Pat. Nos. 5,871,728 and 6,224,859). Evidence that DCs can be used or manipulated to exhibit “tolerogenic” effects in autoimmune disease has come predominantly from studies in experimental models of T1D or multiple sclerosis. (See, for example, Fu et al., 1196, Transplantation 62:659-665; Rastellini et al., 1995, Transplantation 60:1366-1370; Lu et al., 1997, Transplantation 27:1808-1815; Gao et al., 1999, Immunology 98:159-170; Hirano et al., 2000, Transplant Proc. 32:260-264; and Thomson & Lu, 1999, Transplantation 68:1-8). Prevention of spontaneous diabetes in NOD mice can be achieved by transferring in vitro antigen-pulsed syngeneic DCs, IFNγ-treated DCs, CTLA4Ig-treated DCs, or DCs from the pancreatic lymph nodes of diabetic NOD mice. (Lu et al., 1999, J Leukoc. Biol. 66:293-296). The delayed development of diabetes in NOD mice by DC-based therapy has been correlated with 1) increased levels of Th2-type cytokines in pancreatic infiltrating cells and 2) reduced β cell apoptosis. (Khanna, et al., 2000, J. Immunol. 164:1346-1354). Adoptive transfer of auto-Ag (myelin basic protein; MBP)-pulsed thymic DCs or splenic DCs treated with CTLA4Ig prevents the development of experimental autoimmune encephalomyelitis (EAE) in the rat, which is also associated with a Th2 cytokine switch. However, mature DCs can lose their tolerogenicity over time.

Thus, there is a need for tolerogenic DCs that do not readily mature and thus lose their tolerogenicity when introduced into a host.

### SUMMARY OF THE INVENTION

In one aspect, the present invention features tolerogenic dendritic cells (DCs) that have surface antigens DEC205 and B220, but lack surface antigen CD19 (i.e. they are DEC205+ B220+ CD19−). As shown herein, these novel quasi-dendritic, quasi-B cell-like cells exhibit increased immunosuppressive activity in a subject. Notwithstanding these increased immunosuppressive properties, these novel cells can be further engineered to enhance their immunosuppressive activity, for example, by transducing the cells with an agent that results in the down-regulation of an immunostimulatory protein or up-regulation of an immunosuppressive protein.

In another aspect, the invention features methods for enriching for the tolerogenic dendritic cells of the invention from tissue preparations containing the cells. In a preferred embodiment, the cells are enriched for using a method selected from the group consisting of competent-mediated lysis, fluorescent-activated cell sorting (FACS) and metrizamide gradient centrifugation.

In yet a further aspect, the invention features therapeutic methods for using the instant disclosed DCs alone or in conjunction with other immunosuppressive agents to prolong graft survival or prevent or ameliorate transplant rejection. Alternatively, preparations of the cells alone or in conjunction with an immunosuppressive agent may be administered to a subject to treat or prevent the development of an autoimmune disease, including for example, type 1 diabetes, systemic lupus erythrematosis, rheumatoid arthritis, multiple sclerosis, myasthenia gravis, and scleroderma.

That the isolated cells of the invention are naturally present in humans indicates that therapeutic use will be associated with a positive safety profile. In addition, the ability of these cells to remain intact for at least about 20 weeks, support their therapeutic efficacy.

Other features and advantages of the invention will be apparent based on the following Detailed Description and claims.

### DESCRIPTION OF THE DRAWINGS

FIG. 1A is a photomicrograph showing clusters of cells propagated from normal B10 (H-2^{b}) liver non-parenchymal cells (NPC) with IL-3 and anti-CD40 mAb after 48 hours in culture. Panel A.

FIG. 1B is a photomicrograph showing the immunocytochemical staining of liver NPC-derived cells for MHC class II (I-A^{b}) expression of cells cultured for 6 days that display typical DC morphology of long, thin, and beaded processes.

FIG. 2A is a transmission electron micrograph showing the ultrastructure of liver NPC-derived cells propagated with IL-3 and anti-CD40 mAb for 6 days that demonstrate numerous, extensive cytoplasmic processes, irregularly shaped nuclei, numerous mitochondria, and a paucity of paracrystalline cytoplasmic granules (bars, 1 μm).

FIG. 2B is a scanning electron micrograph of NPC-derived liver cells propagated with IL-3 and anti-CD40 mAb for 3 days that display veils (bars, 1 μm).

FIG. 2C is a scanning electron micrograph of NPC-derived liver cells propagated with IL-3 and anti-CD40 mAb for 5 days that display veils (bars, 1 μm).

FIG. 3A are a series of histograms showing the flow cytometric analysis of cell surface Ag expression on liver-derived DEC205⁺ B220⁺ CD19⁻ cells propagated with IL-3 and anti-CD40 mAb (filled histograms) compared with liver granulocyte-macrophage colony stimulating factor (GM-CSF DC; open histograms). Appropriate Ig isotype controls are shown as dotted profiles.

FIG. 3B are a series of graphs depicting the distribution of liver DEC205⁺ B220⁺ CD19⁻ cells that double stain with phycoerythrin-conjugated (PE) anti-DEC205 mAb and fluorescein-conjugated (FITC) anti-B220 mAb or FITC anti-CD19 mAb. The data are representative of three separate experiments.

FIG. 4A is an autoradiogram showing the results of DNA PCR analysis for Ig gene rearrangements in liver-derived DEC205⁺ B220⁺ CD19⁻ cells. DNA was isolated from CD11c⁺ DCs propagated in GM-CSF and IL-4 (BM-IL-4 DCs) (lane 1), or liver DEC205⁺ B220⁺ CD19⁻ cells (lanes 2 and 3, representing samples from two experiments) purified by flow cytometry. Lane 4, DNA ladder. Thymus and spleen cells are represented in lanes 5 and 6. A RAG-deficient pro-B cell line 63-12 is shown in lane 7. Lane 8, Cell-free negative control. D to J_{H}, V to DJ_{H}, and V to J₇₈ gene rearrangements were identified in DEC205⁺ B220⁺ CD19⁻ cells.

FIG. 4B is a series of graphs showing flow cytometric analysis of Ig expression on liver DEC205⁺ B220⁺ CD19⁻ cells double stained with PE anti-B220 mAb or PE anti-DEC205 mAb and FITC anti-CD19 mAb, FITC anti-IgG mAb, FITC anti-IgM mAb, FITC anti-Ig_{κ} mAb, or FITC anti-Igλ mAb. The data are representative of three separate experiments.

FIG. 5A is a graph showing tritiated thymidine ([³H]TdR) uptake by T cells in mixed leukocyte reaction (MLR). C3H(H-2 ^{k}) splenic T cells were cultured with γ-irradiated B10 (H-2^{b}) spleen cells, mature myeloid DCs (BM IL-4 DC), immature myeloid DCs (liver-derived DCs propagated in GM-CSF, hereinafter referred to as GM-CSF DCs), or liver-derived DEC205⁺ B220⁺ CD19⁻ cells for 3 days at various stimulator:responder (S:R) ratios.

FIG. 5B is a photomicrograph showing in situ nick-end labeling (TUNEL) cytocentrifuge preparations of T cells from 3-day culture with either DEC205⁺B220⁺CD19⁻cells (at a T:DC ratio of 10:1), BM IL-4 DCs, or liver GM-CSF DCs (original magnification, ×100).

FIG. 5C is a graph showing the restoration of allostimulatory activity of liver-derived DEC205⁺B220⁺CD19⁻cells by addition of the common caspase inhibitor zVAD-fmk (100 μM) at the beginning of 3-day MLR. Results are expressed as mean cpm ±SD of triplicate cultures and are representative of three experiments.

FIG. 6A is a histogram showing the presence of CD4⁺regulatory T cells in animals treated with bone marrow-derived DCs.

FIG. 6B is a histogram showing an increase in CD4⁺regulatory T cells in animals treated with liver-derived DEC205⁺B220⁺CD19⁻DCs.

FIG. 6C is a graph showing the distribution of CTLA4⁺/CD25⁺cells among the gated CD4⁺cells in animals treated with bone marrow-derived DCs.

FIG. 6D is a graph showing the distribution of CTLA4⁺/CD25⁺cells among the gated CD4⁺cells in animals treated with liver-derived DEC205⁺B220⁺CD19⁻DCs.

FIG. 7A is a series of graphs showing cytokine levels in supernatants from 3-day MLR were assayed by enzyme-linked immunosorbent assay (ELISA) in which C3H(H-2^{k}) splenic T cells were cultured with γ-irradiated B10 (H-2^{b}) BM IL-4 DC, liver GM-CSF DC, or liver-derived DEC205⁺B220⁺CD19⁻cells at a S:R ratio of 1:10 for 2-4 days. Data are expressed as pg/ml±1 SD from triplicate cultures.

FIG. 7B is a series of graphs the flow cytometry analysis of cytokine profiles of C3H splenic T cells that were cultured with γ-irradiated B 10 liver-derived DEC205⁺B220⁺CD19⁻cells at a S:R ratio of 1:10 for 3 days, stained with FITC anti-IL-10 Ab and PE anti-IFN-γ. Results are representative of three separate experiments.

FIG. 8A are a series of graphs showing the cytokine profiles of either B10 BM IL-4 DC (a), liver GM-CSF DC (b) or liver-derived DEC205⁺B220⁺CD19⁻cells (c) with or without LPS stimulation and then cultured with or without the addition of LPS (10 μg/ml, for an additional 48 h of culture). IL-10, IL-12, TNF-β, IFN-γ, and nitric oxide (NO) were measured in culture supernatants by ELISA (or colorimetric assay based on the Griess reaction for NO). Results are expressed as mean picograms per milliliter for cytokines, and micromolar for NO±SD of triplicate experiments.

FIG. 8B is an autoradiogram showing cytokine mRNA expression in DC was determined by RNase protection assay from either B 10 BM IL-4 DC (a), liver GM-CSF DC (b) or liver-derived DEC205⁺B220⁺CD19⁻cells (c) with or without LPS stimulation and then cultured with or without the addition of LPS (10 μg/ml, for an additional 48 h of culture). Results are representative of three separate experiments.

FIG. 9 is a series of histograms showing the flow cytometric analysis of gated CD3⁺cells after culturing C3H(H-2^{k}) splenic T cells were cultured with γ-irradiated B10 (H-2^{b}) BM IL-4 DCs, liver GM-CSF DCs, or liver-derived DEC205⁺B220⁺CD19⁻cells at S:R ratios of 1:10 for 1-3 days and double stained with TUNEL and anti-CD3. Cells incubated with label solution in the absence of terminal transferase served as controls (open histograms). Results are representative of three separate experiments.

FIG. 10 is a photomicrograph showing the migration and survival of B10 (H-2^{b}) liver-derived DEC205⁺B220⁺CD19⁻cells in allogeneic recipients 2 days after a total of 2×10⁵ sorted DEC205⁺B220⁺CD19⁻cells were injected into the hind footpad of C3H(H-2^{k}) mice and cryostat sections of spleen were stained with donor-specific anti-1-A^{b} mAb, wherein cells bearing donor I-A^{b} Ag localized to the white pulp, mainly in the T cell-dependent region in proximity to the central arteriole (original magnification, ×400). The Inset is a higher magnification (×1000) photomicrograph of cells bearing I-A^{b} Ag.

FIG. 11 is a graph showing that the administration of liver-derived DEC205⁺B220⁺CD19⁻cells significantly prolongs cardiac allograft survival in a donor-specific fashion after a total of 2×10⁶ B10 mature myeloid DCs (BM IL-4 DCs), immature myeloid DCs (liver GM-CSF DCs), or liver-derived DEC205⁺B220⁺CD19⁻cells were injected i.v. into C3H(H-2^{k}) recipients 7 days before transplantation of a vascularized cardiac graft from B 10 (H-2^{b}) or BALB/c (H-2^{d}, third-party) donor (n=6 in each group).

FIG. 12A is a photomicrograph showing the hematoxylin and eosin (H&E) stained section of a pancreatic islet from a non-obese diabetic (NOD) mouse at 12 weeks of age.

FIG. 12B is a photomicrograph showing the anti-CD4 mAb stained section of a pancreatic islet non-obese diabetic (NOD) mouse at 12 weeks of age.

FIG. 12C is a photomicrograph showing the anti-CD8 mAb stained section of a pancreatic islet non-obese diabetic (NOD) mouse at 12 weeks of age.

FIG. 12D is a photomicrograph showing the anti-CD11b mAb stained section of a pancreatic islet non-obese diabetic (NOD) mouse at 12 weeks of age.

FIG. 12E is a photomicrograph showing the anti-CD11c mAb stained section of a pancreatic islet non-obese diabetic (NOD) mouse at 12 weeks of age.

FIG. 12F is a photomicrograph showing the anti-CD45R mAb stained section of a pancreatic islet non-obese diabetic (NOD) mouse at 12 weeks of age.

FIG. 13A is a photomicrograph showing the hematoxylin and eosin (H&E) stained section of a pancreatic islet from a NOD mouse treated with liver-derived DEC205+B220+CD19−DC at 30 weeks of age.

FIG. 13B is a photomicrograph showing the anti-CD4 mAb stained section of a pancreatic islet from a NOD mouse treated with liver-derived DEC205+B220+CD19−DC at 30 weeks of age.

FIG. 13C is a photomicrograph showing the anti-CD8 mAb stained section of a pancreatic islet from a NOD mouse treated with liver-derived DEC205+B220+CD19−DC at 30 weeks of age.

FIG. 13D is a photomicrograph showing the anti-CD11c mAb stained section of a pancreatic islet from a NOD mouse treated with liver-derived DEC205+B220+30 CD19−DC at 30 weeks of age.

FIG. 13E is a photomicrograph showing the anti-CD11b mAb stained section of a pancreatic islet from a NOD mouse treated with liver-derived DEC205+B220+CD19−DC at 30 weeks of age.

FIG. 13F is a photomicrograph showing the anti-CD45R mAb stained section of a pancreatic islet from a NOD mouse treated with liver-derived DEC205+B220+CD19−cells at 30 weeks of age.

FIG. 14A is a graph showing the an increase in insulitis over time in NOD mice, as quantitated according to Salomon et al., 2000, Immunity 12:431-440.

FIG. 14B is a graph showing that the administration of liver-derived DEC205⁺B220⁺CD19⁻cells significantly lowers the amount of insulitis present in 30 week-old NOD mice relative to 18 week-old untreated control NOD mice.

FIG. 15 is a graph showing the prevention of the development of Type 1 Diabetes (T1D) in NOD mice by the administration of liver-derived DEC205⁺B220⁺CD19⁻cells.

FIG. 16 is a graph showing the increase of ovalbumin-specific (OVA) T cells in draining lymph nodes of mice treated with OVA-pulsed dendritic cells at days 2-3 following delivery of liver-derived DEC205⁺B220⁺CD19⁻cells to the mice.

FIG. 17 is a series of histograms and graphs showing that liver-derived DEC205⁺B220⁺CD19⁻cells stimulate markers of apoptosis of T cells in NOD mice than mice treated with bone marrow-derived DCs.

### DETAILED DESCRIPTION OF THE INVENTION

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “autoimmune disease” is used herein to refer to conditions in which lymphocytes, especially T cells, respond to self-antigens in a manner that compromises tissue integrity or causes tissue damage. Non-limiting examples of autoimmune diseases include: alopecia aerata, artherosclerosis, asthma, autoimmune myocarditis, autoimmune diabetes, insulin-dependent (Type 1) diabetes, diabetic periodontitis, diabetic retinopathy, and diabetic nephropathy, Graves' disease, Graves ophthalmopathy, glomerulonephritis, lichen sclerosis, glomerulonephritis, multiple sclerosis, myasthenia gravis, obesity (non-diabetic or associated with diabetes), psoriasis, rheumatoid arthritis, scleroderma, septic shock, sleep disorders and chronic fatigue syndrome, systemic lupus erythematosus, systemic sclerosis, thyroid diseases (e.g. goiter and struma lymphomatosa (Hashimoto's thyroiditis, lymphadenoid goiter), as well as AIDS.

The term “dendritic cell” is used herein to refer to a type of antigen presenting cell (APC) that is essential for initiation of primary immune responses and the development of tolerance. DCs express major histocompatibility complexes (MHC) that are necessary for stimulation of T cell populations.

An “effective amount” is an amount sufficient to effect a beneficial or desired clinical result upon treatment. An effective amount can be administered to a patient in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition and the form and effective concentration of the antigen-binding fragment administered.

The terms “host” and “subject” are used herein interchangeably to refer to mammalian (including human) recipients of transplants or to mammalians who suffer from an autoimmune disease.

The term “immunostimulatory” is used herein to refer to increasing overall immune response.

The term “immunostimulatory protein” is used herein to refer to a protein that is involved with the initiation or progression of an immune response is an subject. Examples include cytokines, such as interleukin 12 (IL-12) and tumor necrosis factor α (TNF-α).

The term “immunosuppressive” is used herein to refer to reducing overall immune response.

The term “immunosuppressive agent” is used herein to refer to any agent that when administered to a subject reduces the subjects overall immune response. Examples include small molecule drugs such as, azathioprine (Imuran), tacrolimus (FK506), cyclosporin (CSA, CyA, Sandimmune, or Seoral), cyclophosphamide (Cytoxan), daclizumab (Denapax), mycophenolate mofetil (CellCept, RS, or RS-61443), prednisone (Deltasone), and sirolimus (rapamycin or rapamune); proteins (including polypeptides and peptides), such as muromonab-CD3 (Orthoclone OKT3, or OKT3), and cytokines, such as interleukin 4 (IL-4), interleukin-6 (IL-6), interleukin10 (IL-10), interferon γ, macrophage migration inhibitory factor (MIF), lymphotoxin β (LTB) and transforming growth factor β (TGFβ); nucleic acids (including ribonucleic acids, deoxyribonucleic acids and analogs thereof in single stranded (including sense or antisense) or double stranded form as well as ribozymes), lipids and carbohydrates.

The term “propagated” is used herein to refer to cells removed from mammalian tissue and maintained in vitro.

The term “substantially enriched” is used herein to refer to a solution containing at least two times, three times, four times, ten times, or one hundred times the concentration of liver-derived DEC205⁺B220⁺CD19⁻cells than other cells.

The term “substantially purified” is used herein to refer to preparations of DEC205⁺B220⁺CD19⁻cells having less than about 20% (by dry weight) contaminating cells, preferably less than about 5% contaminating cells, and more preferably less than about 1% contaminating cells.

The term “tolerogenic” is used herein to refer to induced antigen-specific unresponsiveness.

The term “transplant” as used herein includes tissue grafts or cell grafts, including homografts or allografts, which are derived from the same species as the host, or xenografts, which are derived from a species different from the host.

Tolerogenic Dendritic Cells

The invention is based, at least in part, on the discovery of novel populations of dendritic cells that exhibit enhanced tolerogenic properties that have been enriched from human and mouse tissue. Consistent with their classification as DCs, these cells exhibit DC morphology and express the DC marker DEC 205 (Swiggard et al., 1995, Cell. Immunol. 165:302-311). However, contrary to typical DCs, these cells express the B220 antigen (Ag), which is a marker of cell activation typically associated with B cells. However, these cells do not exhibit all features of B cells, as they do not express the B cell-restricted Ag CD19. Thus, these cells are characterized by their features in common with both DCs and B cells in their novel profile of DEC205+, B220+, and CD19−.

In addition to the expression of a surface antigen usually associated with B cells, these DEC205+, B220+, and CD19− DCs undergo Ig gene rearrangement and expression that corresponds to that occurring relatively early in B cell development. Although these cells have characteristics in common with B cell precursors or immature B cells, DEC205+, B220+, and CD19− DCs express high levels of MHC class II antigen, which is usually associated with mature DCs. The high level of expression of MHC class II antigen expression distinguishes these cells from other tolerogenic DCs, for example those described in U.S. Pat. Nos. 5,781,728 and 6,224,859.

In addition to having unique characteristics, DEC205+, B220+, and CD19− DCs are particularly well-suited for immunosuppressive therapeutics. In Examples 2, 3, 4, 6, and 7, the DEC205+, B220+, and CD19− DCs exhibit superior tolerogenic properties over previously described tolerogenic DCs.

Method of Enriching for and Propagating Cells

The DEC205+B220+CD19− DCs can be obtained by (a) harvesting cells from mammalian tissues, (b) substantially depleting the cells with non-DCs, including T cells, B cells, NK cells, granular cells, and macrophages by competent-dependent lysis and/or metrizamide gradient centrifugation, and (c) incubating the cells with recombinant IL-3 and anti-CD40 mAb. (See Materials and methods). Alternatively, the DEC205+, B220+, and CD19− DCs can be obtained by (a) harvesting cells from mammalian tissues and (b) using FACS with anti-DEC205 mAb, anti-B220 mAb, and anti-CD19 mAb. Further, the DEC205+, B220+, and CD19− DCs can be obtained by (a) treating an animal with the fins-like tyrosine kinase 3 ligand (Flt-3L), (b) harvesting the cells from mammalian tissues, and (c) performing metrizamide gradient centrifugation. The tissues from which tolerogenic DCs are preferably isolated from, but are not limited to liver, spleen, BM, blood, thymus, and lymph nodes.

Prior to harvesting, it may be useful to stimulate the production of dendritic cells in general or specifically stimulate DEC205+, B220+, and CD19− DCs by administering an appropriate agent to the donor animal. For example, it has been shown that administration to mice of a tyrosine kinase receptor ligand, such as fms-like tyrosine-kinase 3 ligand (Flt-3L), as described in Smith et al., 2001, Immunology 102(3):352-8, triggered increased production of dendritic cells in blood and bone marrow. (Maraskovsky et al., 1997, Blood 90 (suppl 1):2585a, 1997).

Once generated, the DCs may be propagated by any suitable cell culturing technique known to the skilled artisan. For example, the DCs may be propagated in accordance with the methods described in Inaba et al., 1992 J. Exp. Med. 176:1693-1702; Lu et al., 1995, Transplantation 60:1539-1545; and Lu et al., 1997, Transplantation 64:1808-1815; and Woo et al., 1994, Transplantation 58:848).

Genetically Engineered DEC205+B220+, and CD19− Cells

Although as shown herein, the novel DEC205+, B220+, and CD19— cells exhibit immunosuppressive properties, these properties may be further enhanced by genetically engineering the cells. For example, the cells can be transduced with vectors that result in increased expression of an immunosuppressive protein, using any of a variety of methods well known to one of skill in the art. For example, increased expression of endogenous genes may be activated by introducing into the DC a new transcription unit, or gene activation construct, that comprises an exogenous regulatory sequence, an exogenous exon, and a splice site, operably linked to the second exon of an endogenous gene, wherein the cell comprises the exogenous exon in addition to exons present in the endogenous gene (see, for example, U.S. Pat. Nos. 5,641,670; 5,773,746; 5,733,761; 5,968,502; 6,702,989; and 6,565,844).

Further, increased expression of immunosuppressive agents may be accomplished by genetically engineering DEC205+B220+CD19— DCs to enhance the expression of the exogenous genes that encode immunosuppressive agents, by way of transgenes, vectors, and the like. The expression of exogenous genes may be accomplished by means of introducing viral-based or non-viral-based vectors into the cell. (See for example U.S. Ser. No. 09/469,519, 10/213,939, P.C.T. Pub. WO 01/83713 or U.S. Ser. No. 09/844,915)

The DEC205+B220+CD19— DCs may be genetically engineered to decrease the expression of immunostimulartory agents by inhibiting the production of nucleic acids or inhibiting the production of polypeptides, using antisense technology, intrabodies, ribozymes, gene silencing techniques, and the like. For instance, the production of IL-12 may be blocked by preventing transcription of the genes encoding the IL-12 p35 or p40 subunits by triplex formation or other means, by preventing translation of the mRNA encoding the IL-12 p35 or p40 subunits by antisense or ribozyme technology, or by expressing an intrabody (an intracellular single-chain antibody) that binds the p35 or p40 subunits of IL-12, thereby preventing their interaction to form the biologically-active heterodimeric form of IL-12. Additionally, the expression of NFKB may be inhibited, for example as described in PCT Publication No. WO/83713, published Nov. 8, 2001.

Methods for introducing and expressing exogenous genes or inhibiting the expression of endogenous genes may be accomplished following, for example, the teachings in the following: U.S. Pat. Nos. 5,176,996; 5,264,564; 5,256,775; and 5,093,246; PCT Publication No. WO88/09810, published Dec. 15, 1988; PCT Publication No. WO89/10134, published Apr. 25, 1988; PCT International Publication WO90/11364, published Oct. 4, 1990; Van der Krol et al., 1988, BioTechniques 6:958-976; Stein et al., 1988, Cancer Res. 48:2659-2668; Wanger et al., 1994, Nature 372:333; Letsinger et al., 1989, Proc. Natl. Acad. Sci. U.S.A. 86:6553-6556; Lemaitre et al., 1987, Proc. Natl. Acad. Sci. U.S.A. 84:648-652; Zon (1988), Pharm. Res. 5:539-549; Perry-O'Keefe et al., 1996, Proc. Natl. Acad. Sci. U.S.A. 93:14670; Eglom et al., 1993, Nature 365:566; Gautier et al., 1987, Nucl. Acids Res. 15:6625-6641; Inoue et al., 1987, Nucl. Acids Res. 15:6131-6148; Inoue et al., 1987, FEBS Lett. 215:327-330; Bernoist et al., 1981, Nature 290:304-310; Yamamoto et al., 1980, Cell 22:787-797; Wagner et al., 1981, Proc. Natl. Acad. Sci. U.S.A. 78:1441-1445; Brinster et al., 1982, Nature 296:39-42; Haseloff and Gerlach, 1988, Nature 334:585-591; Smithies et al., 1985, Nature 317:230-234; Thomas, et al., 1987, Cell 51:503-512; Thompson et al., 1989, Cell 5:313-321; and Goeddel, 1990, Gene Expression Technology: Methods in Enzymology 185, Academic Press, San Diego, Calif.

Therapeutic Uses and Pharmaceutical Compositions of DEC205+B220+CD19− DCs cells

As shown in the following Examples 6 and 7, the tolerogenic DCs of the present invention enhance tolerance in a mammalian subject and are therefore useful for preventing or minimizing transplant rejection and/or prolonging transplant survival in a mammalian host, or alternatively for preventing and/or treating an autoimmune disease.

The tolerogenic DCs described herein can be administered using any physiologically acceptable route, such as, for example, oral, pulmonary, parenteral (intramuscular, intra-articular, intraperitoneal (i.p.), intravenous (i.v.) or subcutaneous (s.c.) injection), inhalation (via a fine powder formulation or a fine mist, aerosol), transdermal, intradermal, nasal, vaginal, rectal, or sublingual routes of administration. The DCs are preferably administered by i.v. or s.c. injection.

The tolerogenic DCs of the present invention may be administered alone or in conjunction with a carrier, which may be any physiologically acceptable solution or dispersion media, such as saline or buffered saline. The carrier may also comprise antibacterial and antifungal agents, isotonic and adsorption delaying agents, and the like. Except insofar as any conventional media, carrier or agent is incompatible with the active ingredient, its use in the compositions is contemplated. The carrier may further comprise one or more immunosuppressive agents in dosage unit form.

The dosage of the tolerogenic DCs of the present invention to be administered in vivo can be determined with reference to various parameters, including the species of the host, the age, weight and disease status. Dosage may also depend upon the location to be targeted within the host, e.g. foreign graft transplantation site or joints of an arthritic host. For example, direct targeting of joints may require different dosages than administration into the blood stream of a mammalian host. The dosage is preferably chosen so that administration causes an effective result, as measured by molecular assays, prolongation of foreign graft survival, and alleviation of an inflammatory disease. Dosages may range from 1×10⁴ DCs to 1×10⁹ DCs per administration, most preferably 5×10⁵ DCs to 5×10⁷ DCs. To achieve maximal therapeutic effect, several doses may be required.

Where the tolerogenic DCs of the present invention are to be used for the prolongation of foreign graft survival or the amelioration of transplant rejection, administration of the DCs into the host may be conducted prior to transplantation with the foreign graft or transplanted tissue. More particularly, administration may be conducted one week prior to transplantation. Prior administration may provide a prophylactic effect. Administration may also be conducted at the time of the transplant and up to one-two weeks after the transplant to ensure acceptance of the foreign graft or transplanted tissue.

Administration of the tolerogenic DCs for the prolongation of graft survival or the amelioration of transplant rejection may occur in combination with one or more immunosuppressive agents. In this treatment regimen, the infusion of DCs may permit a reduction in the dose of immunosuppressant needed to prevent rejection, thereby minimizing the potentially deleterious side-effects of immunosuppression and thus increasing the safety of the procedure. For example, the ability of FK 506 to prolong graft survival, which is improved when FK 506 is administered to the host together with dendritic cells. See Khanna et al., 1998, Transplantation 65:479-485, incorporated herein by reference. Furthermore, CSA inhibits the expression of costimulatory molecules in vivo on dendritic cells. See Lee et al., 1999, Transplantation 68:1255-1263, incorporated herein by reference. Immunosuppressive agents may be administered to a host at the time of foreign graft transplantation and may be administered daily thereafter for a period of time necessary to optimize graft survival. Practitioners will know to adjust the administration of immunosuppressive agents. The amount of immunosuppressive agents necessary may change due to the therapeutic effect of the tolerogenic DCs of the present invention, as well as the host response to the transplantation.

To determine appropriate times for administering the tolerogenic DCs of the present invention, a skilled artisan may employ conventional clinical and laboratory means for monitoring graft survival, graft function and the host's reaction to the transplant. Biochemical and immunological tests may be used for such monitoring. Where the tolerogenic DCs of the present invention are to be used to prevent or ameliorate diseases related to inflammation or autoimmune disease, administration may be conducted daily, weekly, monthly or yearly depending on the alleviation of the symptoms of the disease. Administration can continue as long as necessary to alleviate the disease or to inhibit its onset.

The tolerogenic DCs of the present invention need not be derived from the same species as the host to be treated, although as mentioned above, they may be from the same species from a donor or from the host itself. For instance, DCs may be isolated from a baboon donor to produce the tolerogenic DCs of the present invention and may be administered into a human host to enhance tolerogenicity therein. See e.g., Starzl et al., 1994, Immunological Reviews 141:213-244, the contents of which are incorporated herein by reference.

In another aspect, the invention relates to a kit for use in enhancing tolerogenicity in a host comprising the tolerogenic DCs of the present invention. The DCs may be isolated in accordance with the methods described herein. The kit may contain cells in culture, cells frozen in media plus a cryoprotectant [such as dimethyl sulfoxide (DMSO)], or lyophilized cells. Alternatively, the kit may comprise the reagents necessary to isolate and propagate the tolerogenic DCs. For example, the kit may comprise reagents for isolating precursors from a host, reagents for generating DCs from the precursors, reagents for propagating the DCs, such as at least one cytokine (e.g. GM-CSF), TGF-β or other reagents necessary to enhance or maintain the tolerogenicity of the DCs. The kit may comprise any variation of the reagents necessary to produce the tolerogenic DCs. The kit may additionally comprise a protocol for the administration of the DCs of the present invention, a physiologically acceptable carrier, immunosuppressive agents, etc. The kit may further comprise diagnostic reagents to determine the therapeutic effectiveness of the DCs after administration. The kit may be used to prolong foreign graft survival in a transplant host or to treat an inflammatory related disease in a host.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

The invention now being generally described, it will be more readily understood by reference to the following examples, which are included merely for purposes of illustration of certain aspects and embodiments of the present invention, and are not intended to limit the invention.

Materials and Methods

Animals. Male C57BL/10 (B10; H-2_{b}), C3H(H-2ₖ), BALB/c (H-2^{d}) and non-obese diabetic (NOD) mice were purchased from The Jackson Laboratory (Bar Harbor, Me.) and used at 8-12 wk of age except where otherwise indicated. Animals were maintained in the specific pathogen-free facility of the University of Pittsburgh Medical Center (Pittsburgh, Pa.) and provided with Purina rodent chow (Ralston Purina, St. Louis, Mo.) and tap water ad libitum.

Isolation of NPC from liver. Livers were perfused in situ with collagenase solution, followed by further ex vivo digestion. The NPC fraction was then isolated by centrifugation over a Percoll gradient (Sigma, St. Louis, Mo.), as described previously (Lu et al., 1994, J. Exp. Med. 179:1823-1834).

Propagation of DEC205⁺, B220⁺, CD19⁻cells from livers. Liver NPC were depleted of T, B, NK, granular cells, and macrophages by complement-dependent lysis using a mAb mixture comprising anti-CD3, CD19, NK1.1, CD14, Gr-1 (all Abs from PharMingen, San Diego, Calif.), and low toxicity rabbit complement (Accurate Chemical and Scientific, Westbury, N.Y.). Thereafter, 2×10⁶ lineage-negative cells were cultured in 2 ml of RPMI 1640 (Life Technologies, Gaithersburg, Md.) supplemented with antibiotics and 10% (v/v) FCS (referred to subsequently as complete medium), and mouse rIL-3 (10 ng/ml; BioSource International, Camarillo, Calif.), plus anti-CD40 mAb (2 ng/ml; PharMingen, San Diego Calif.) in flat-bottom 24well culture plates for 5-7 days. Nonadherent cells released from clusters were harvested for further characterization. For comparative purposes, mature myeloid DC propagated from bone marrow (BM) in GM-CSF plus IL-4 (referred to subsequently as BM IL-4 DCs) and immature myeloid DCs propagated from liver NPC in GM-CSF alone (referred to subsequently as liver GM-CSF DCs), as described elsewhere (Lu et al., 1994, J. Exp. Med. 179:1823-1834; Lu et al., 1995 Transplantation 60:1539-1545), were used. Briefly, BM cells or liver NPCs were cultured in 24-well plates (2×10⁶/well) in complete medium containing both mouse rGM-CSF (4 ng/ml) and rIL-4 (1000 U/ml) (both from Schering-Plough, Kenilworth, N.J.) or GM-CSF alone for 5-7 days. The selection and purification procedures were similar to those reported initially by Inaba et al. (Inaba et al., 1992, J. Exp. Med. 176:1693-1702) and modified by Lu et al. (Lu et al., 1994, J. Exp. Med. 179:1823-1834; Lu et al., 1995 Transplantation 60:1539-1545).

A similar method to that used in mouse was used to propagate DEC205⁺, B220⁺, CD19⁻cells from human livers. When propagating human DEC205+, B220+, CD19- cells, however, the cells were incubated with anti-human IL-3 mAb and anti-human CD40 mAb, in stead of to use anti-mouse mAbs.

In some cases, the DC cell population was substantially enriched either after or in the absence of MLR using metrizamide gradient centrifugation. 2 ml metrizamide gradient prepared from complete medium at the concentration of 15.2% (w/v) was placed as the bottom of a tube, and the cell suspension (8 mL) is added on the top. The tube was then centrifuged at 600 g for 15 min. The cells at the interface of the two solutions are collected and washed twice. The collected cells are substantially enriched in DEC205⁺B220⁺CD19⁻cells

Flow cytometry. Cell surface Ag expression was analyzed by cytofluorography using an Epics Elite flow cytometer (Coulter, Hialeah, Fla.). FITC- or PE-conjugated mAbs were obtained from PharMingen, except for anti-DEC-205 mAb (generously provided by R. M. Steinman, The Rockefeller University, New York, N.Y., but now available commercially through BD Biosciences). For intracellular cytokine detection, cells were incubated in brefeldin A (10 μg/ml; Sigma) for 5 h, then washed with 1% saponin/1% FCS/PBS, as described previously (Khanna et al., 2000, J. Immunol. 164:1346-1354). Double staining was performed using FITC- or PE-conjugated anti-H-2 K^{b}, anti-IL-10, or anti-IFN-γ mAbs. Cells were then washed with 1% FCS/PBS and resuspended in 1% formaldehyde before analysis. Appropriate isotype- and species-matched irrelevant mAbs were used as controls.

Detection of apoptosis. T cells were stained with PE-conjugated anti-CD3ε, anti-CD4, or anti-CD8α mAb, and DNA strand breaks were identified by TUNEL. Following surface CD3, CD4, or CD8 staining, cells were fixed in 4% paraformaldehyde and permeabilized with 0.1% Triton X-100 and 0.1% sodium citrate. TUNEL reaction mixture of the Cell Death Detection kit (Roche Diagnostics, Indianapolis, Ind.) was then added according to the manufacturer's instructions. Cells incubated with label solution in the absence of terminal transferase were used as negative controls. Quantitative analysis was performed by flow cytometry, with 5000 events acquired from each sample. For identification of apoptotic cells in cytospin preparations, T cells activated by DC were processed for immunocytochemical detection of incorporated biotin-dUTP by peroxidase-labeled avidin, followed by an enzyme reaction using aminoethylcarbazole as the substrate, as described elsewhere (Lu et al., 1996, J. Immunol. 157:3577-3586).

Mixed leukocyte reaction (MLR). To determine the allostimulatory capacity of DCs, one-way MLR were performed with γ-irradiated (20 Gy) DCs or spleen cells from B10 (allogeneic) or C3H (syngeneic) mice as stimulators and nylon wool-purified C3H spleen T cells (2×10₅) as responders. Cultures (200 μl) were established in triplicate in 96-well round-bottom microculture plates and maintained in complete medium in 5% CO₂ in air at 37° C. for 3-4 days. [³H]TdR (1 μCi/well) was added for the final 18 h of culture, and incorporation of [³H]TdR into DNA was assessed by liquid scintillation counting in an automated counter. Results are expressed as mean cpm±1 SD. In apoptosis inhibition experiments, a common caspase inhibitor peptide, benzyloxycarbonyl-Val Ala-Asp-fluoromethyl ketone (zVAD-fmk; Alexis, San Diego, Calif.), was added (100 μM) at the beginning of the MLR culture. DMSO served as a control.

Cytokine and NO quantitation. IL-2, IFN-γ, IL-4, IL-10, IL-12, TNF-α, and TGF-β levels in supernatants of MLR or DC cultures were quantitated using ELISA kits (BioSource International), with sensitivity limits of 20-25 pg/ml, as described (Khanna et al., 2000, J. Immunol. 164:1346-1354). A standard curve using recombinant cytokine was generated for each assay. NO levels were determined by the colorimetric Griess reaction that detects the stable end product nitrite, as described (Lu et al., 1996, J. Immunol. 157:3577-3586).

RNase protection assay. Total RNA was extracted from DCs by the guandinium isothiocyanate-phenol-chloroform method using TR1 reagent (Sigma), as described (Chomczynski et al., 1987, Anal. Biochem. 162:156-159). Cytokine mRNA expression was determined using the RiboQuant multiprobe RNase protection assay system (PharMingen) following the manufacturer's instruction. Briefly, 5 μg of total RNA was hybridized to ³²P-labeled RNA probes overnight at 56° C., followed by treatment with RNase for 45 min at 30° C. The murine L32 and GAPDH riboprobes were used as controls. Protected fragments were submitted to electrophoresis through a 7 M urea/5% polyacrylamide gel and then exposed to Kodak X-OMAT film (Kodak, Rochester, N.Y.) for 72 h.

DNA PCR assay for Ig rearrangement. DNA was prepared for PCR by lysing cells in 200 μl of PCR lysis buffer (10 mM Tris, pH 8.4, 50 mM KCl, 2 mM MgCl₂, 0.45% Nonidet P-40, 0.45% Tween 20, and 60 μg/ml proteinase K), incubating them at 55° C. for 1 h, and then inactivating the protease by heating to 95° C. for 10 min. DNA at a concentration of 5000 genomes/μl was used for PCR. PCR (50 μl) were performed as described previously (Schlissel et al., 1991, J. Exp. Med. 173:711-720). Thirty cycles of amplification were performed, after which one-fifth of each reaction was analyzed on a 1.4% agarose gel in Tris-borate buffer. The gel was blot transferred to a nylon membrane and probed with ³²P-labeled DNA from the appropriate Ig C region. Primer sequences are as published elsewhere (Schlissel et al., 1991, J. Exp. Med. 173:711-720). Germline alleles were detected using a primer (Mu0) 322 nt 5′ to J_{H}1. D_{H} R and L primers are oligonucleotide mixtures degenerate at two and three positions, respectively, and homologous to all members of the Dfl16 and Dsp2 D gene families. D to J rearrangements were detected as amplified fragments of˜1033, ˜716, or ˜333 nt depending on whether J_{H}1, J_{H}2, or J_{H}3 was rearranged. To detect V to DJ rearrangements, a mixture of three different degenerate oligonucleotides homologous to conserved framework region 3 sequences of three V_{H} gene families (V_{H}7183, V_{H}558, and VHQ52) and the J3 primer was used. This resulted in amplified VDJ rearrangements of ˜1058, ˜741, or ˜358 nt. PCR products were detected by hybridization with appropriate Ig gene probes. Both DJ and VDJ rearrangements resulted in loss of Mu0 sequence and its amplification product. V to DJ rearrangement events resulted in loss of all of the D_{H} L primer target sequences and amplified DJ fragments.

In vivo migration. Cells propagated from B10 mice were injected s.c. (5×10⁵ cells in 50 μl) into a hind footpad of normal allogeneic C3H recipients. Animals were sacrificed in groups of three at days 1, 2, 3, and 7 after injection. The draining popliteal lymph node, thymus, and spleen were removed, embedded in Tissue-Tek OCT compound (Miles, Elkart, Ind.), and frozen at −80° C. Cryostat sections (4 μm) were air dried at room temperature overnight for further processing.

Immunocytochemistry. B 10 MHC class II⁺cells were identified in cryostat sections or cytospin preparations using biotinylated mouse IgG2a anti-mouse I-A^{b} (PharMingen) in an avidin-biotin-alkaline phosphatase complex (ABC) staining procedure. Isotype- and species-matched irrelevant mAb were used as controls. Donor MHC class II+(1-A^{b+}) cells were counted in 100 high-power fields, and the data were expressed as number of 1-A^{b+} cells per high-power field.

Heterotopic vascularized heart transplantation. Surgical procedures were performed under methoxyflurane (Medical Development, Springvale, Australia) inhalation anesthesia. Cardiac anastomoses to the abdominal aorta and inferior vena cava were performed as described previously (Ono and Lindsey, 1969, J Thorac. Cardiovasc. Surg. 7:225-229). The function of the donor heart was monitored daily by abdominal palpation. Rejection was defined as total cessation of contraction, which was confirmed by histological examination.

Statistical analyses. Graft survival times between groups of transplanted animals were compared using the Mann-Whitney U test. A p<0.05 was considered to be statistically significant.

### Example 1

### Description of DEC205⁺B220⁺CD19⁻Cells from Liver

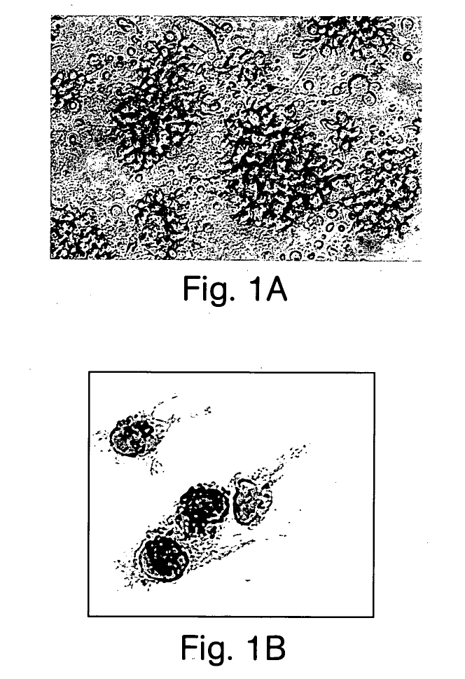

Generation of DEC205⁺B220⁺CD19⁻cells from liver NPCs. NPCs were isolated from livers of B10 mice. Approximately 7-8×10⁶ cells were obtained from each liver, with <5% hepatocyte contamination. An Ab mixture and complement were used to deplete CD4⁺, CD8α⁺, CD14⁺, CD19⁻, NK1.1+, and Gr-1⁺cells. Cells were then cultured in complete medium containing IL-3 for 3-4 days. FIG. 1A shows that clusters of proliferating cells were noted in the cultures. The addition of anti-CD40 mAb induced the formation of long dendritic process on these cells. Following 3-4 additional days in culture, the cells detached from the adherent clusters. By 6-8 days, ˜3×10⁶ such cells were obtained from each mouse liver.

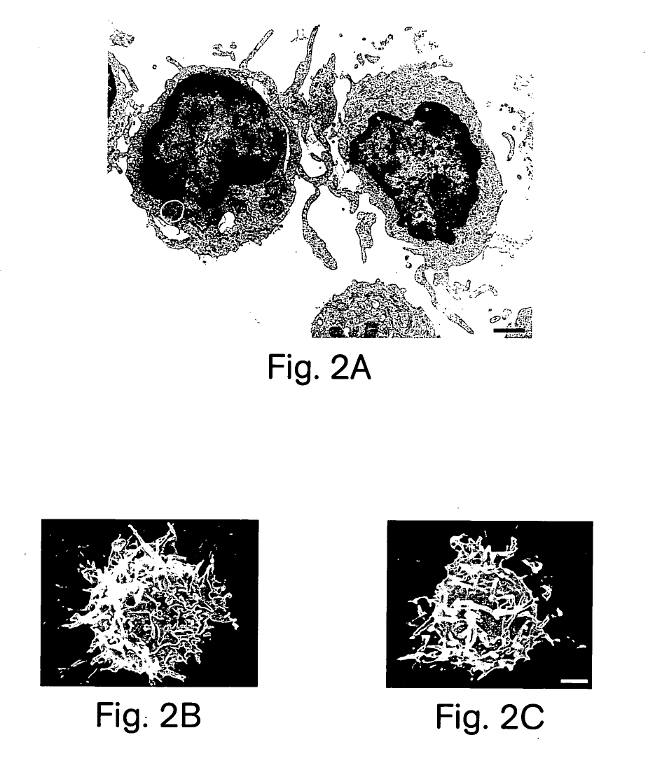

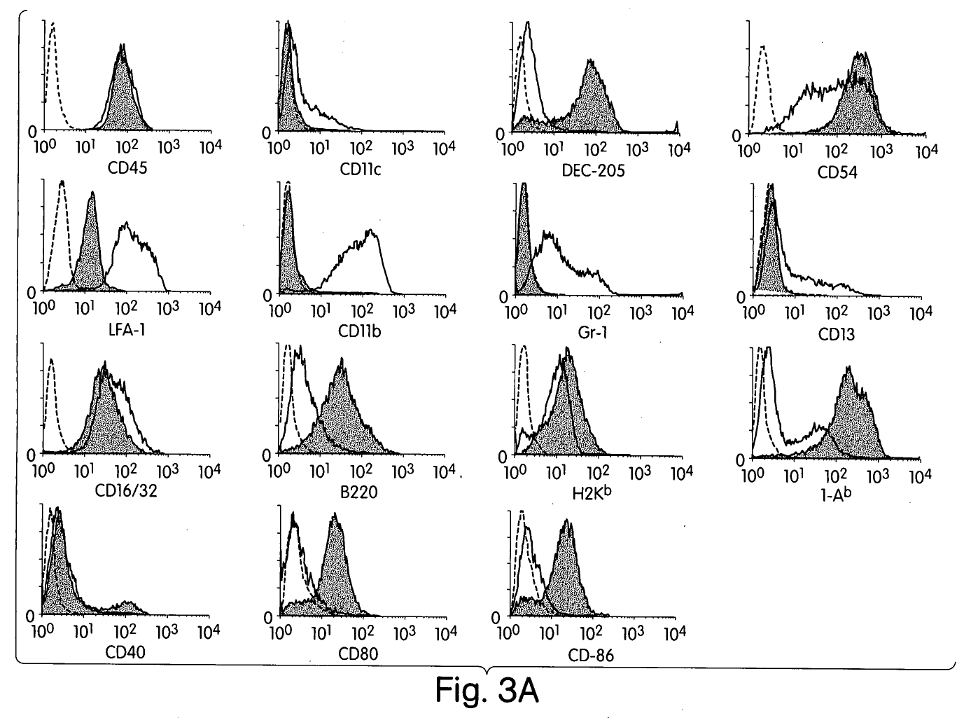

FIG. 1B shows that, morphologically, the cells displayed characteristics of DC, including irregular-shaped eccentric nuclei, a paucity of prominent cytoplasmic granules, and extended dendrites. Transmission electron microscopy, as shown in FIG. 2A, further delineated the delicate cytoplasmic processes, an abundance of mitochondria, and the absence of electron-dense granules. The typical dendritic veils or pseudopodia were observed under scanning electron microscopy, as shown in FIG. 2, panels B and C.

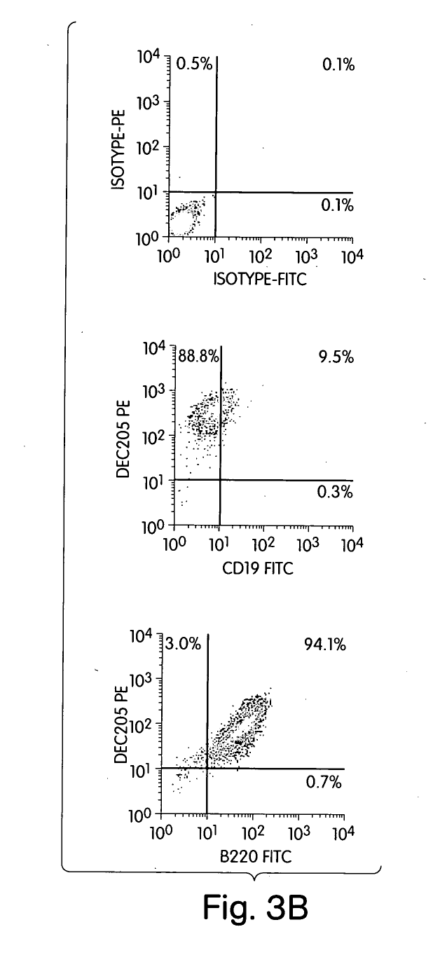

Immunophenotypic analysis demonstrated high expression of CD45, MHC class I, MHC class II, costimulatory molecules (CD40, CD80, and CD86), and the lymphoid DC marker DEC--205, as shown in FIG. 3A. The myeloid DC marker CD11c was absent. Additionally, the cells did not express Ags associated with myeloid cells (CD13, CD11b, or CD14), T cells (CD3ε, CD4, and CD8α), or NK cells (NK1.1). Of interest, the cells expressed B220, an Ag typically present on B cells, but lacked the B cell-restricted molecule CD19, as shown in FIG. 3B). By contrast, FIG. 3A shows that liver GM-CSF DCs expressed the myeloid lineage molecules CD11b, CD13, and CD14, as well as the myeloid DC marker CD11c, as described (Lu et al., 1994, J. Exp. Med. 179:1823-1834). FIG. 3A also shows that these cells exhibited only low levels of DEC-205. MHC and costimulatory molecule expression also were low, which was consistent with an immature phenotype. Further stimulation with anti-CD40 mAb, Flt-3 ligand, or extracellular matrix protein induced partial or full maturation (Lu et al., 1994, J. Exp. Med. 179:1823-1834; Drakes et al., 1997, J. Immunol. 159:4268-4278.). Thus, two distinct subsets of cells bearing molecules associated with Ag presentation can be propagated from precursors present in liver NPC in response to different cytokines (IL-3/CD40 ligand vs. GM-CSF). BM IL-4 DCs showed a mature mycloid DC phenotype, as reported (Lu et al., 1995, Transplantation 60:1539-1545).

DEC205⁺B220⁺CD19⁻cells propagated from human liver displayed similar morphological and immunophenotypic profile as DEC205⁺B220⁺CD19⁻cells propagated from mouse.

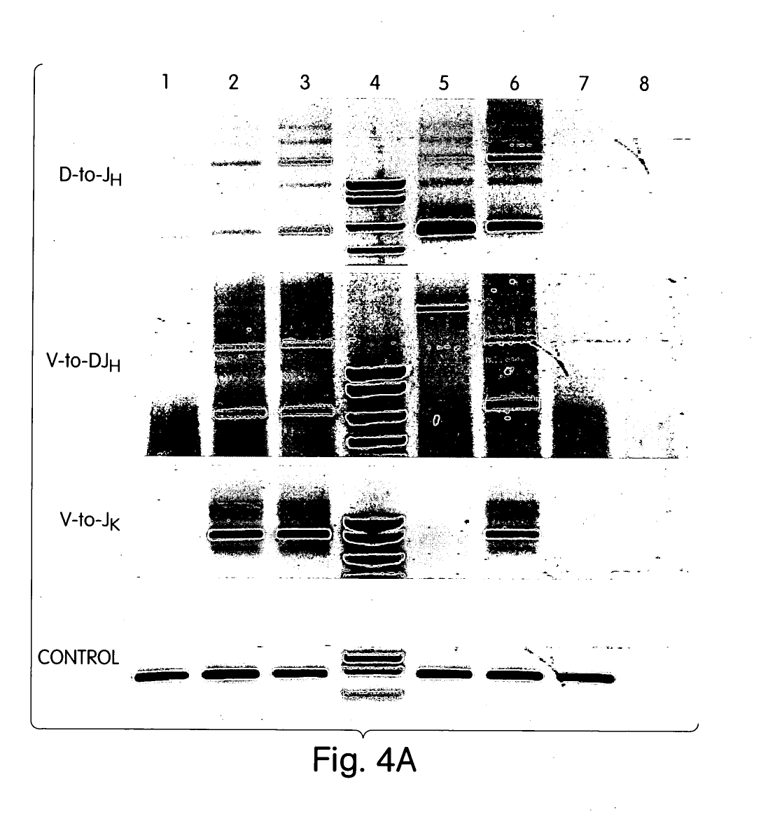

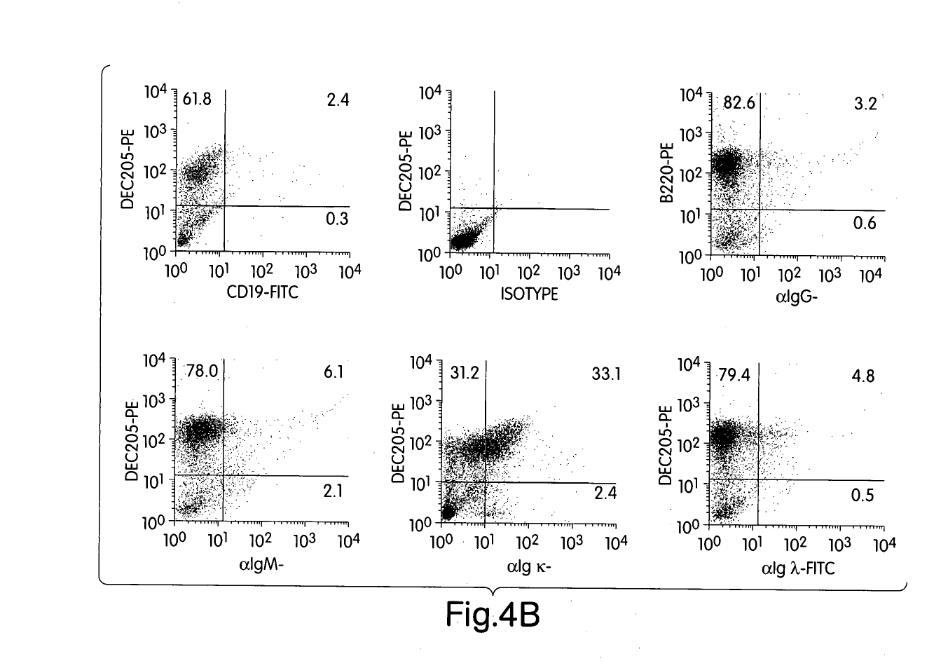

Ig gene rearrangement and expression in liver-derived DEC205⁺B220⁺CD19⁻cells. The expression of B220 by cells derived from mouse liver NPCs in response to IL-3 and CD40 ligation raises the possibility that they derive from B cells. Rearrangement of Ig genes occurs relatively early in B cell development, and are detectable in all but the earliest precursors. DNA was isolated from purified liver-derived DEC205⁺B220⁺CD19⁻cells (sorted by flow cytometry to achieve purity >99%), and analyzed by PCR for Ig gene rearrangements. FIG. 4A shows that D to J_{H}, V to DJ_{H}, and V to Jκ gene rearrangements were identified in the cells. A similar pattern was noted in splenocytes rich in mature B cells. In contrast, myeloid DC had only Ig heavy chain DJ rearrangements. Many T cells and other myeloid lineage cells have similar rearrangements. However, the myeloid DCs lack VDJ and VJκ alleles, as also shown in FIG. 4A.

To determine expression of Ig proteins, the DEC205⁺B220⁺CD19⁻cells were stained with mAbs specific to mouse IgG, IgM, Igκ, or Igλ. The cells lacked expression of IgG, IgM, and Igλ. FIG. 4B shows that a small proportion of cells expressed low levels of Igκ λ (FIG. 4B). Expression of κ light chain is normally found in immature B cells in conjunction with μ-heavy chain to form IgM (Janeway Jr. et al., 1999, In Immunobiology Vol. 6:195). These data indicate that DEC205⁺B220⁺CD19⁻cells may have developed from B cell precursors (pro-B or pre-B), but have arrested or diverged at some point before becoming immature B cells.

### Example 2

### Effects of Liver-derived DEC205⁺B220⁺CD19⁻Cells on Immune Function

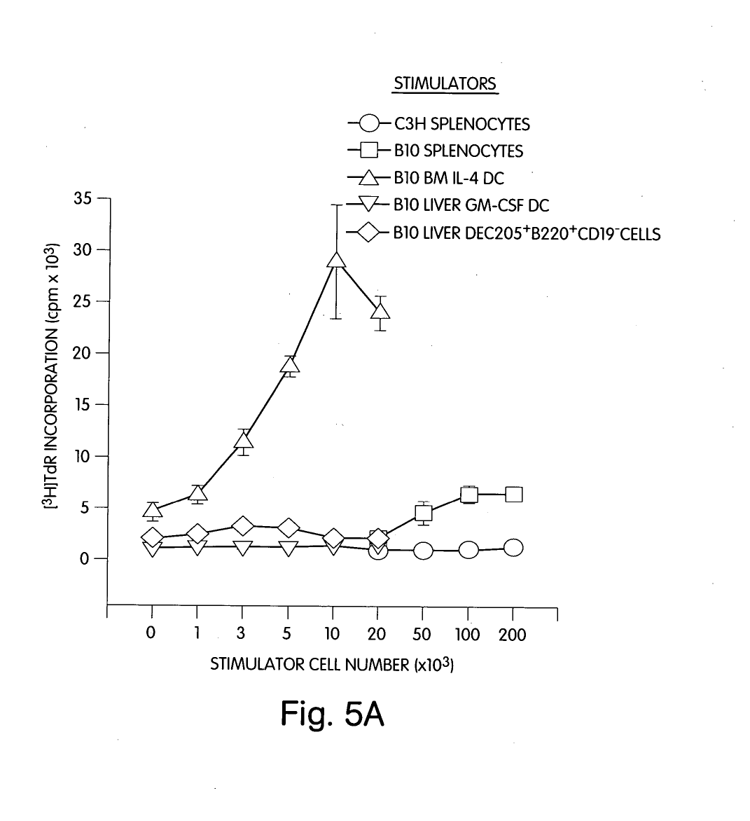

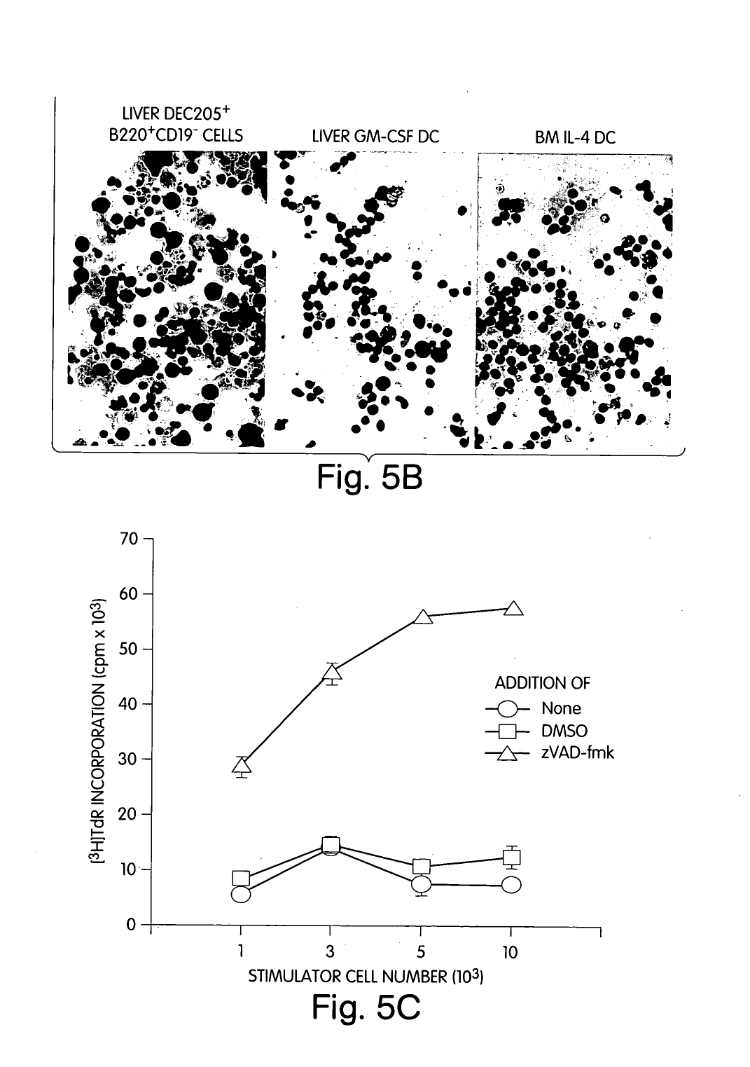

Allostimulatory capacity of liver-derived DEC205⁺B220⁺CD19⁻cells. The allostimulatory capacity of DEC205⁺, B220⁺, CD19⁻cells derived from B 10 liver NPCs was determined in a one-way MLR, performed as described above. Mature mycloid DCs (BM IL-4 DCs) stimulated vigorous allogeneic T cell proliferation, whereas liver-derived DEC⁺B220⁺CD19⁻cells induced very little T cell proliferation, as determined by thymidine uptake. FIG. 5A shows that the allostimulatory capacity was similar to that seen with immature myeloid DCs propagated from liver in response to GM-CSF (liver GM-CSF DCs) (Lu et al., 1994, J. Exp. Med. 179:1823-1834). Low T cell proliferation after stimulation by liver GM-CSF DCs was expected due to low expression of MHC and costimulatory molecules, as shown in FIG. 3A (Lu et al., 1994, J. Exp. Med. 179:1823-1834). However, FIG. 3A also shows that the DEC205⁺B220⁺CD19⁻cells expressed high levels of these molecules, and would be expected to stimulate a brisk T cell response. Despite the high expression of MHC and costimulatory molecules, liver-derived DEC205⁺B220⁺CD19⁻cells display low allostimulatory capacity.

DEC205⁺B220⁺CD19⁻cells propagated from human liver displayed similar low allogeneic T cell responses in vitro as DEC205⁺B220⁺CD19⁻cells propagated from mouse.

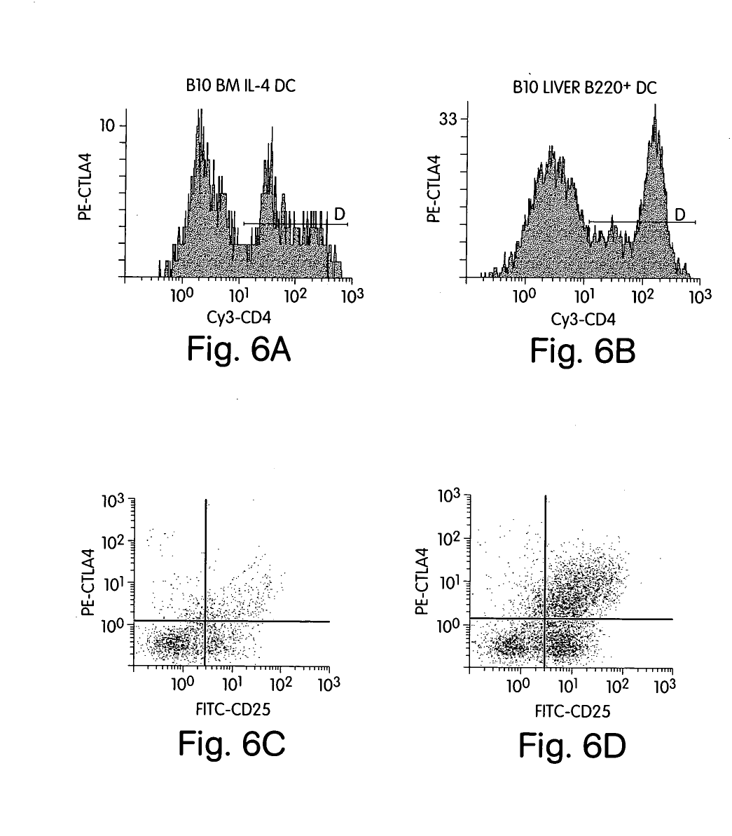

Liver-derived DEC205⁺B⁺CD19⁻cells induce T cell hyporesponsiveness. Injection of DEC205⁺B220⁺CD19⁻cells in naive allogeneic recipients leads to an increase in CD4⁺CD25⁺CTLA4⁺T cells (FIG. 6). For example, as shown in FIG. 6 (upper panels), the administration of liver-derived DEC⁺B220⁺CD19⁻cells from mouse into naive allogeneic recipients increased the population of CD4⁺DCs by approximately 3-fold. FIG. 6, lower panels, shows that the proportion of cells positive for both CTLA4 and CD25 was also significantly increased in the DEC205⁺B220⁺CD19⁻cells-treated animals.

### Example 3

### Effects of DEC205⁺B220⁺CD19⁻cells on T cell differentiation

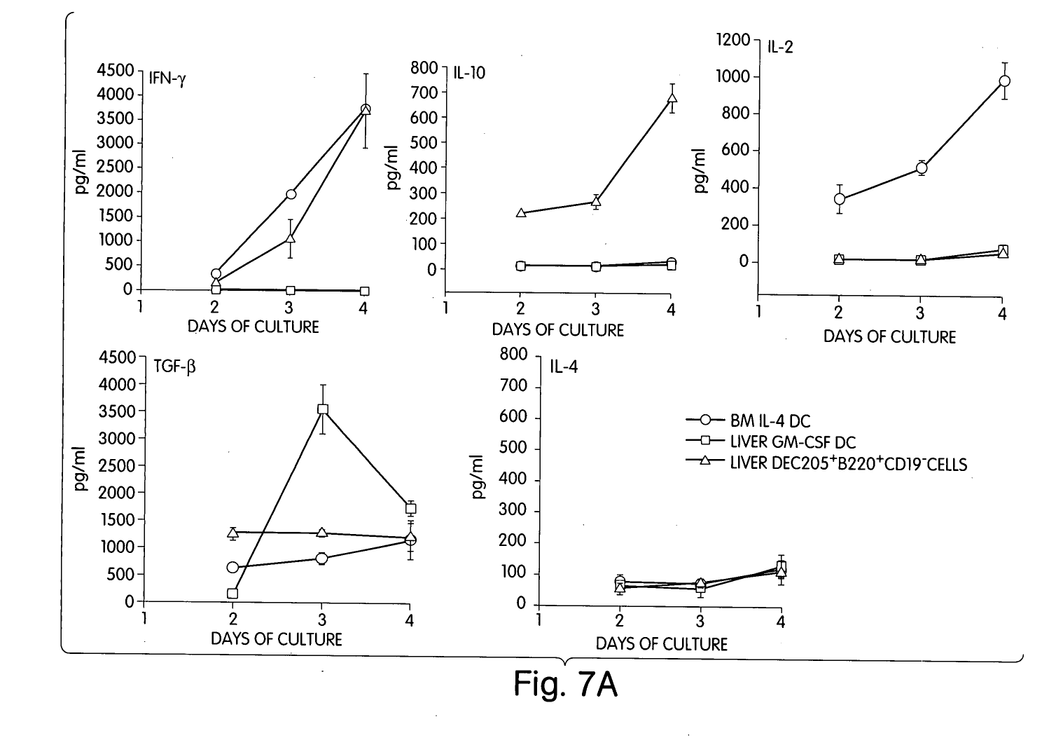

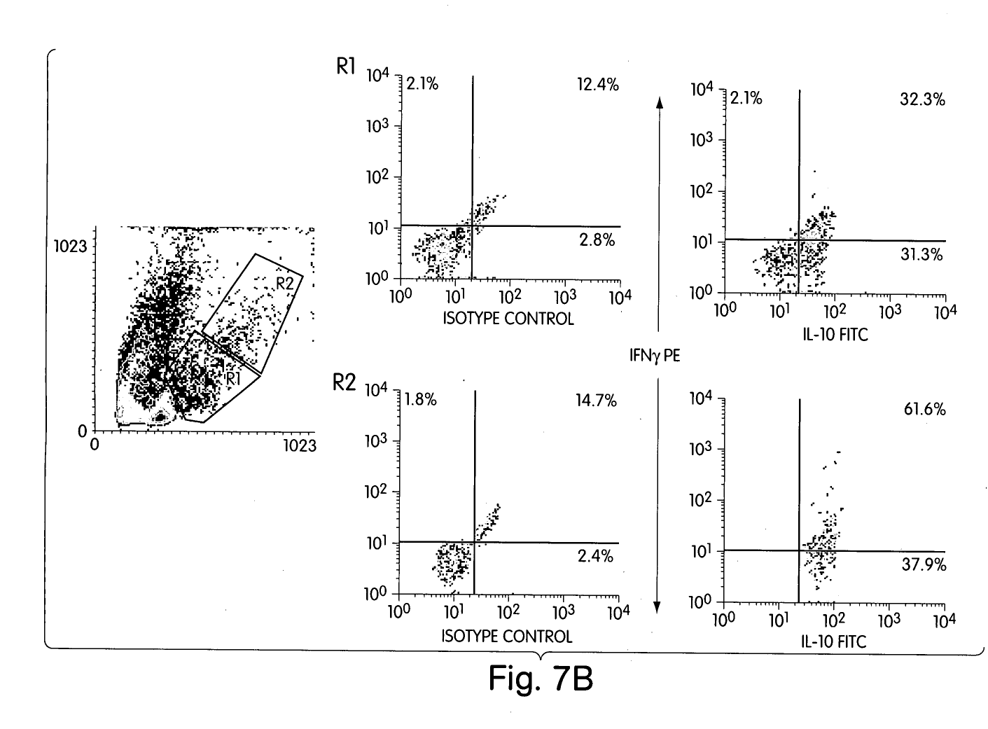

Liver-derived DEC205⁺B⁺CD19⁻cells induce Tr differentiation. T cell differentiation following interaction with various subsets of allogeneic DCs was determined by measuring cytokine levels in the supernatants of 2- to 4-day MLR by ELISA. T cells cultured with BM IL-4 DCs (mature myeloid DCs) from mouse secreted typical Th1 cytokines, including IFN-γ and IL-2, as shown in FIG. 7A. T cells stimulated by liver GM-CSF DCs (immature myeloid DCs) from mouse produced TGF-β, with only low levels of IL-2, IL-4, and IL-10, and no IFN-γ, a profile consistent with Th3 differentiation (Inobe et al., 1998, Eur. J. Immunol. 28:2780-2790). In contrast, T cells were driven by liver-derived DEC205⁺B220⁺CD19⁻cells from mouse to release large amounts of IL-10 and IFN-γ, moderate amounts of TGF-β, and very little IL-2 or IL-4. Such a cytokine profile resembles that of Tr1 cells (Groux et al., 1997, Nature 389:737-742; Saloga et al., 1999, Int. Arch. Allergy Immunol. 118:210-211; Roncarolo et al., 2001, J. Exp. Med. 193:5-9). Cytokine production by T cells was further confirmed by flow cytometric analysis at a single-cell level. Initially, cells were double stained to detect H-2^{k}(responder T cell MHC class I) and cytokine. This revealed that the vast majority of cells producing IL-10 or IFN-γ were T cells (H-2^{k+}; data not shown). Multiple-color staining for IL-10 and IFN-γ allowed identification of a population of cells producing both cytokines. FIG. 7B shows that by narrowing the gate to include unusually large cells (gate R2), a high proportion (>60%) of gated C3H(H-2⁺) T cells stimulated by liver-derived DEC205⁺B220⁺CD19⁻cells released both IFN-γ and IL-10 (FIG. 7B), a pattern resembling Tr1 cells (Groux et al., 1997, Nature 389:737-742; Saloga et al., 1999, nt. Arch. Allergy Immunol. 118:210-211; Jonuleit et al., 2000, J. Exp. Med. 192:1213-1222; Roncarolo et al., 2001, J. Exp. Med. 193:5-9).

Cytokine production by liver DEC205⁺B220⁺CD19⁻cells. Cytokines produced by DCs play a critical role in T cell differentiation (Steinman, 1991, Annu. Rev. Immunol. 9:271-296; Rissoan et al., 1998, Science 283:1183-1186). The cytokine production of liver DEC205⁺B220+⁺CD19⁻cells from mouse compared with mature and immature mycloid DC was also investigated. CD11c⁺BM IL-4 DCs and liver GM-CSF DCs, as well as liver DEC205⁺B220+⁺CD19⁻cells were purified by flow sorting (99% purity), and cultured for 48 h in a resting state or after activation with LPS. Cytokine levels in the supernatants were assessed by ELISA. In the resting state, all three types of APCs produced low levels of cytokines and NO. LPS stimulation induced cytokine production in all types of APCs, but with a distinctly different pattern for each subset. BM IL-4 DCs released large amounts of IL-12, TNF-α and NO, and moderate amounts of IFN-γ. Liver GM-CSF DCs responded to LPS stimulation by markedly increased production of NO and TNF-α, with less pronounced increases in IL-1 2, IFN-γ, and IL-10 production. Thus, upon activation by LPS, the myeloid DCs, in particular mature myeloid DCs, released a characteristic cytokine pattern capable of inducing Th1 differentiation.

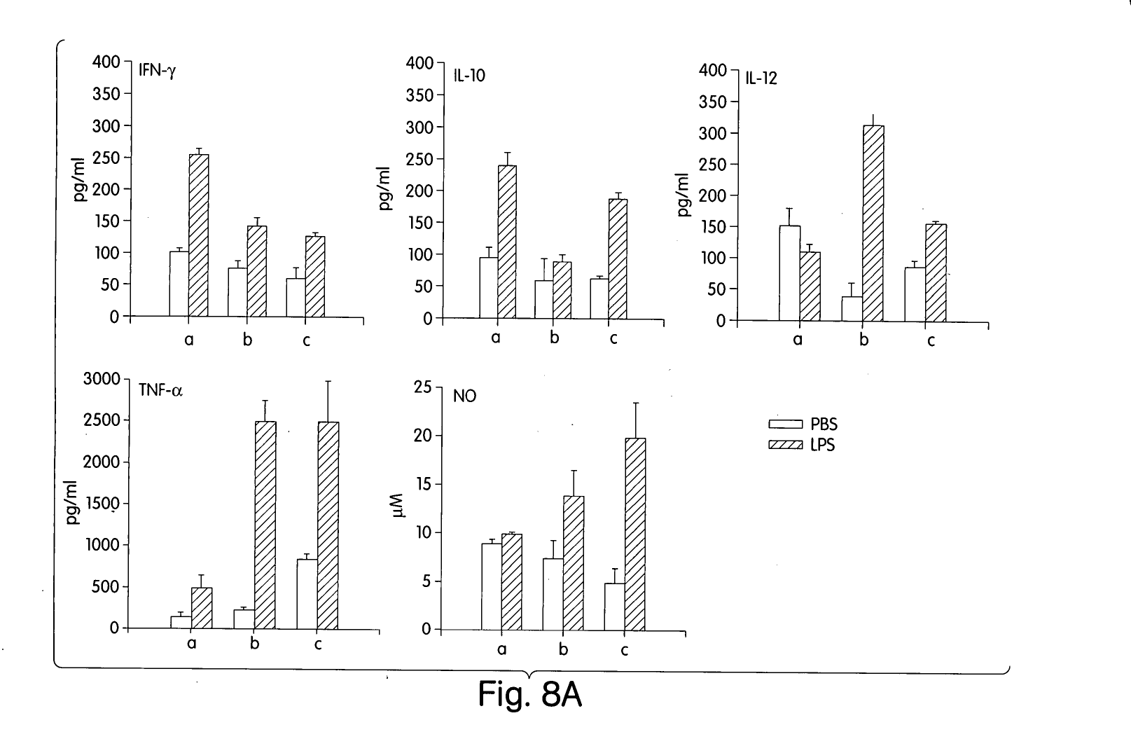

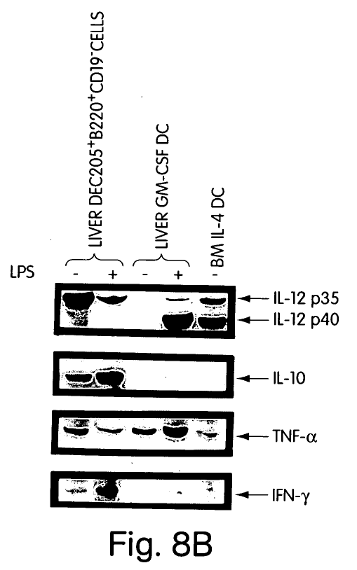

In contrast, as shown in FIG. 8A, liver-derived DEC205⁺B220⁺CD19⁻cells secreted large amounts of IL-10 and IFN-γ in response to LPS. TNF-α, IL-12, and NO production were not induced. This cytokine pattern (high IL-10 and IFN-γ, low IL-12) may be conducive to Tr1 development. Cytokine mRNA expression in these cells was consistent with ELISA results. FIG. 8B shows that liver-derived DEC205⁺B220⁺CD19⁻cells expressed message for the p35 subunit of IL-12, but lacked expression of IL-12 p40. Biological function of IL-12 requires the expression of both subunits (Gubler et al., 1991, Proc. Natl. Acad. Sci. USA 88:4143-4147). Both p35 and p40 mRNA were detected in BM IL-4 DC and inducible in liver GM-CSF DC (FIG. 8B). These results indicate that the signals necessary for T cell differentiation can be provided by the APCs alone, independent of additional exogenous signals. Mature myeloid DCs express cytokines favoring ThI differentiation, whereas liver-derived DEC205⁺B220⁺CD19⁻cells release cytokines promoting Tr cell polarization.

### Example 4

### Effects of DEC205⁺B22⁰+CD19⁻cells on T cell apoptosis

Liver-derived DEC205⁺B22⁰+CD19⁻cells induce T cell apoptosis. In studies of the allostimulatory capacity of liver-derived DEC205⁺B220⁺CD19⁻cells, direct inspection of the T cells over the course of the MLR revealed evidence of T cell death. After 2 days in culture, similar levels of T blasts developed in response to BM IL-4 DC and liver-derived DEC205⁺B220⁺CD19⁻cells, both sets of cells from mouse. Few blasts were observed in T cells responding to liver GM-CSF DC. However, T cells stimulated by liver-derived DEC205⁺B220⁺CD19⁻cells rapidly died, as determined by in situ TUNEL staining. After 3 days in culture, a large number of apoptotic cells mixed with large blast cells were visible in T cells stimulated by liver DEC205⁺B220⁺CD19⁻cells, as shown in FIG. 5B. In contrast, few T cells stimulated by BM IL-4 DCs or liver GM-CSF DCs exhibited evidence of cell death. The low thymidine uptake by T cells stimulated by liver-derived DEC205⁺B220⁺CD⁻cells, despite the appearance of T cell blasts, must therefore be due to rapid apoptosis of activated T cells. Indeed, inhibition of apoptosis by addition of the caspase inhibitor peptide zVAD-fmk (Pronk et al., 1996, Science 271:808-810) restored T cell proliferation induced by liver-derived DEC205⁺B220⁺CD19⁻cells, as shown in FIG. 5C.

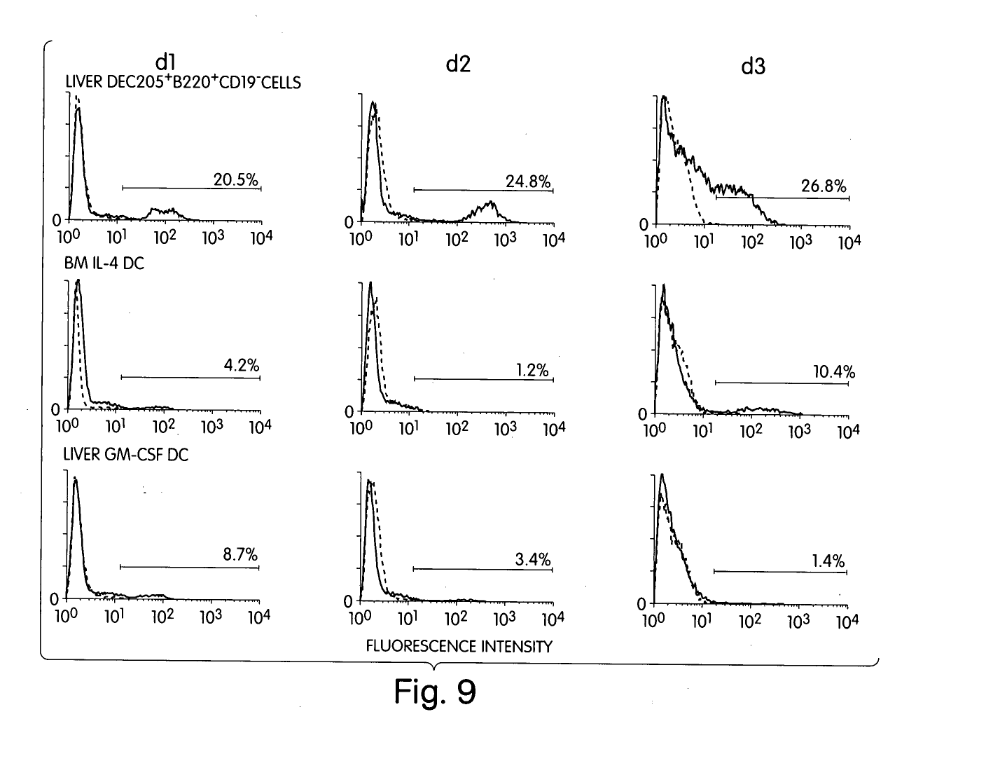

To confirm that T cells stimulated by allogeneic liver DEC205⁺B220⁺CD19⁻cells undergo apoptosis, C3H splenic T cells cultured with various DC subtypes from B10 donors for 1-3 days were stained by TUNEL and anti-CD3, anti-CD4, or anti-CD8 mAbs. Two-color flow cytometric analysis of T cells (CD3⁺) cultured with allogeneic liver DEC205⁺B220⁺CD19⁻cells demonstrated extensive apoptosis (TUNEL⁺) (˜20-30%) as early as 18 h after stimulation, which increased over the next 72 h. BM IL-4 DC or liver GM-CSF DC induced apoptosis in<5-10% of allogeneic T cells, as shown in FIG. 9. Cells were double stained with TUNEL and anti-CD4 or anti-CD8 mAbs to determine the subset of T cells undergoing apoptosis. Liver-derived DEC205⁺B220⁺CD19⁻cells induced similar levels of apoptosis in both CD4⁺and CD8⁺T cells (data not shown).

### Example 5

### Migratory Capacity of Liver-derived DEC205⁺B220⁺CD19⁻Cells

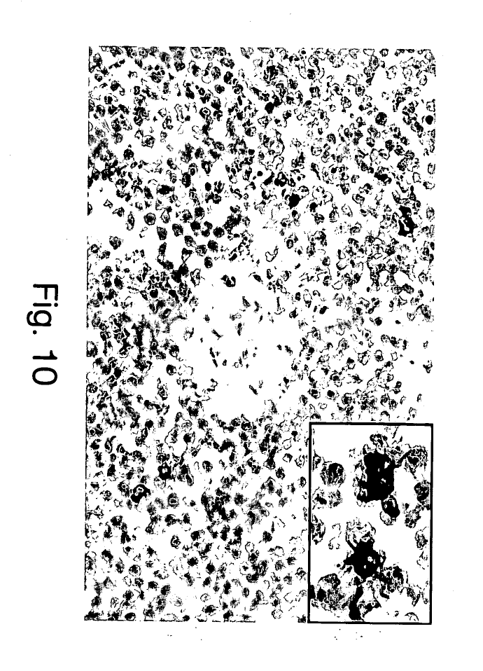

Migration of liver-derived DEC205⁺B220⁺CD19⁻cells. DCs resident in tissue traffic to draining lymph nodes after Ag processing or inflammatory stimuli, presumably to present Ag to lymphocytes. The in vivo migration pattern of DEC205⁺B220⁺CD⁺cells derived from mouse liver was also examined. A total of 5×10₅ purified B 10 liver DEC205⁺B220⁺CD19⁻cells were injected into the footpad of allogeneic C3H recipients. Donor-derived cells were identified by immunocytochemistry utilizing mAb specific to donor MHC class II (1-Ab). I-A^{b+}cells were visible in draining popliteal lymph nodes 1-2 days after injection, but rapidly disappeared after that. Subsequently, I-A^{b+}cells became detectable in the spleen, located predominantly in T lymphocyte-dependent areas in close proximity to arterioles, as shown in FIG. 10. Thus, liver-derived DEC205⁺B220⁺CD19⁻cells exhibit a similar homing ability to that described for mature myeloid DCs (BM IL-4 DCs) and immature myeloid DCs (liver GM-CSF DCs) (Lu et al., 1994, J. Exp. Med. 179:1823-1834; Thomson et al., 1995, Transplantation 59:544-551).

### Example 6

### Effects of liver-derived DEC205⁺B220⁺CD19⁻cells on graft survival

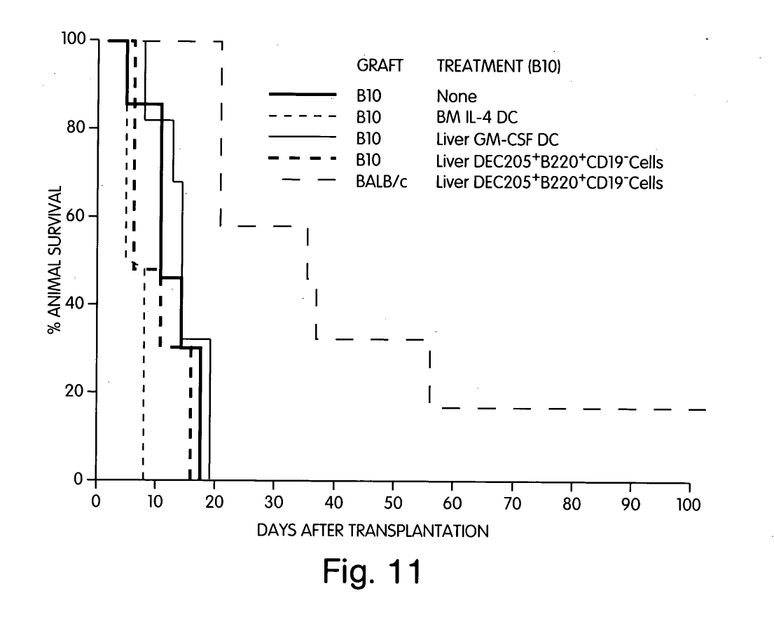

Administration of DEC205⁺B22⁰+CD19⁻cells prolongs cardiac allograft survival. The ability of liver-derived DEC205⁺B220⁺CD19⁻cells to induce T cell apoptosis and promote T cell differentiation consistent with a Tr phenotype suggests that they may play a role in limiting the immune response or maintaining tolerance in vivo. This was assessed in a vascularized cardiac allograft model. A total of 2×10⁶ DEC205⁺B220⁺CD19⁻cells propagated from B 10 liver NPCs were injected i.v. at various time points before B 10 heart transplantation into C3H recipients. Mature myeloid DCs (BM IL-4 DCs), high in costimulatory molecule and MHC expression, and immature myeloid DC (liver GM-CSF DCs), deficient in costimulatory molecule expression, were similarly injected for comparison. As shown in FIG. 11, administration of liver-derived DEC205⁺B22⁰+CD19⁻cells significantly prolonged cardiac allograft survival (median survival time (MST) 37 days, compared with MST 10.5 days in nontreated controls,p<0.05). The optimal time of administration of these cells was 7-10 days before transplantation. Two of six grafts achieved long-term survival (>100 days), with evidence for systemic donor-specific tolerance, as exhibited by acceptance of a subsequent donor skin graft. The effects of liver-derived DEC205⁺B22⁰+CD19⁻cells were donor specific, as they failed to prolong survival of BALB/c cardiac allografts. As previously reported, administration of BM IL-4 DCs exacerbated rejection of cardiac allografts (MST 5 days, p<0.05 compared with nontreated controls). Liver GM-CSF DCs slightly, but not significantly, prolonged allograft survival, as also shown in FIG. 11.

### Example 7

### Effects of liver-derived DEC205⁺B220⁺CD19⁻cells on diabetes

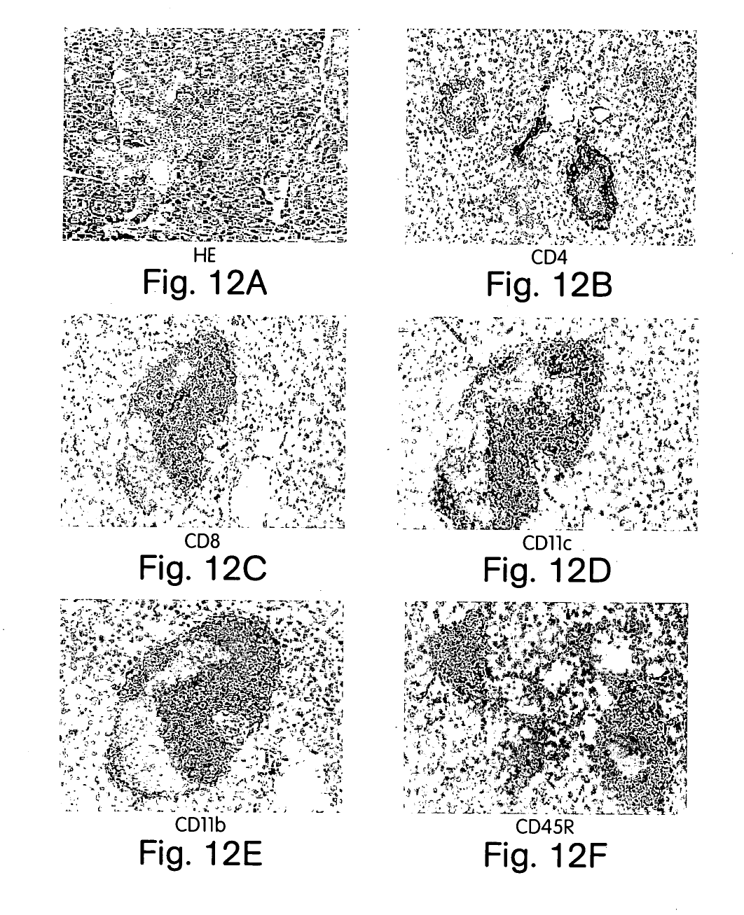

Pathological evidence of diabetes development in NOD mice. The ability of liver-derived DEC205⁺B22⁰+CD19⁻cells to influence the onset of autoimmune diabetes was assessed in NOD mice. To establish a baseline for these studies, histopathological studies were first performed to document the onset of Type 1 diabetes (TID) in these animals. The onset of TID occurs at approximately 6-8 weeks of age in NOD mice and is complete by approximately 20 weeks of age. The proportion of female animals developing T ID during these period ranges from 80% to 95%. Early inflammatory changes in the peri-insulitis are characterized by infiltration of macrophages and DCs. FIG. 12 further shows that CD4⁺and CD8⁺T cells also infiltrate islets at later stages of the disease, as do B cells. Intra-islet destructive infiltration is correlated with increased numbers of DCs and CD8⁺cells, indicating involvement of DCs and cellular immune responses in spontaneous T1D in NOD mice.

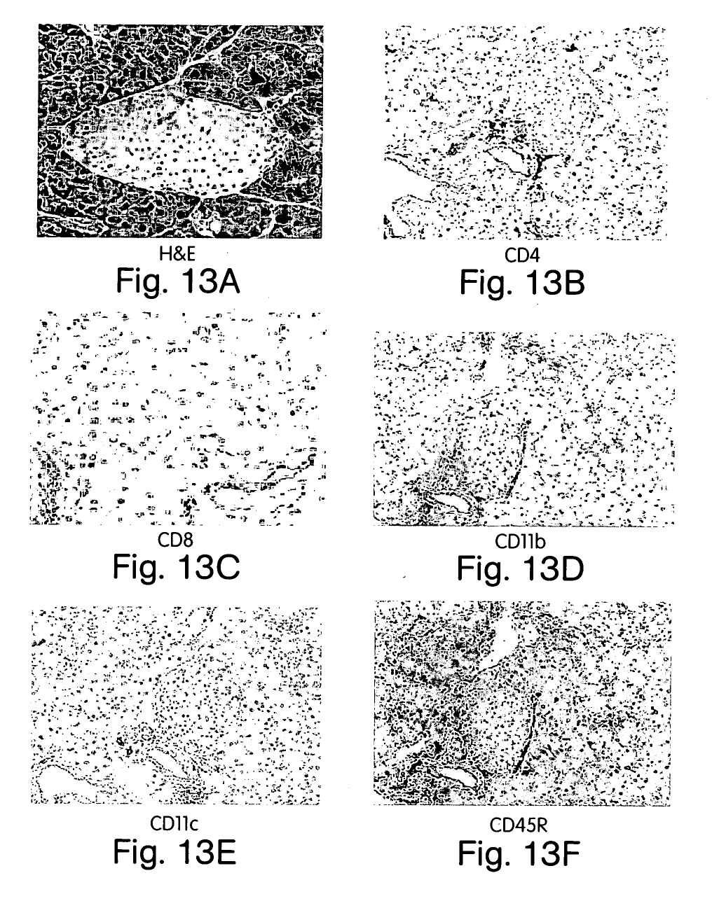

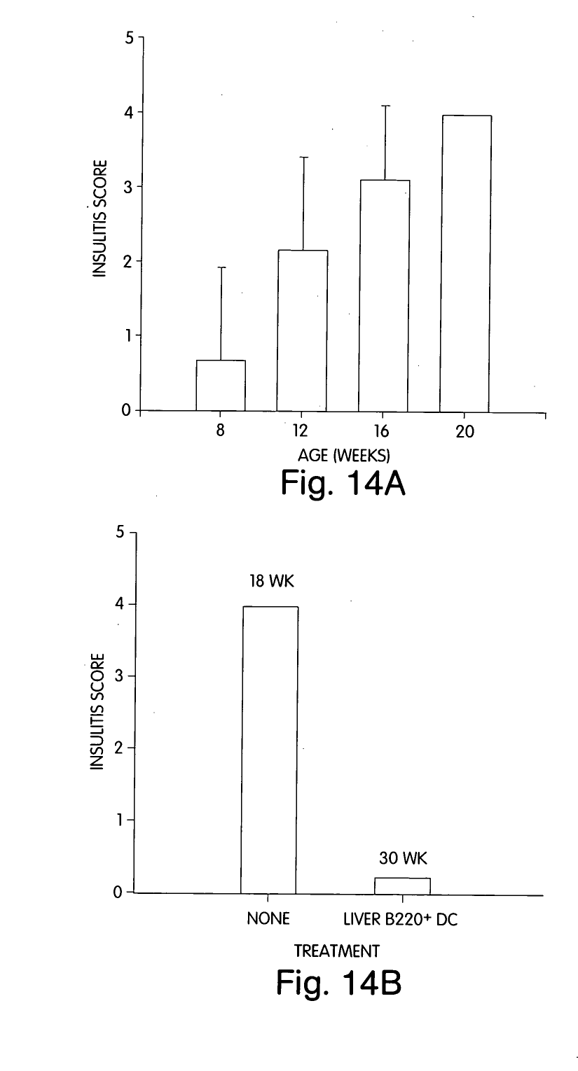

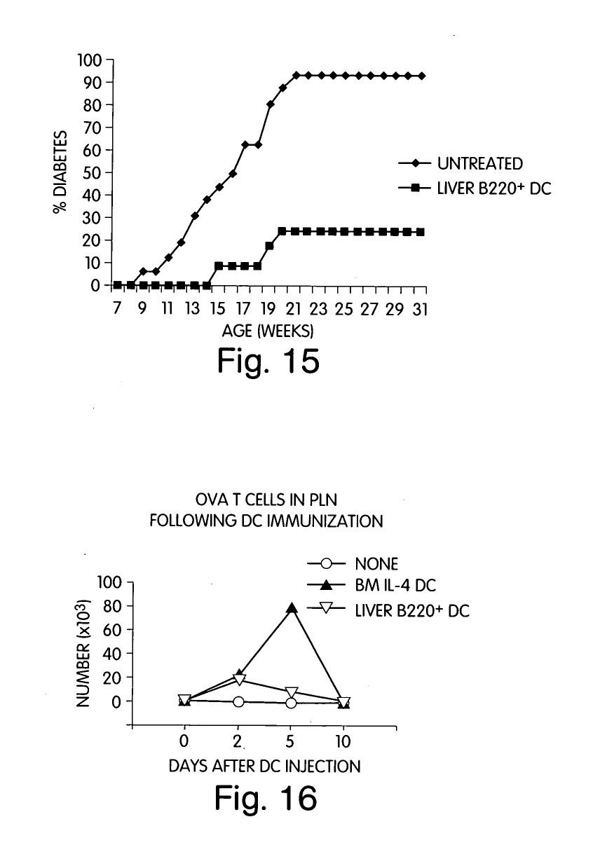

Prevention of diabetes in NOD mice by liver-derived DEC205⁺B22⁰+CD19⁻cells. Administration (i.v,) of propagated liver-derived DEC205⁺B22⁰+CD19⁻cells (2×106) into NOD mice at 11 week of age dramatically precluded the development of diabetes; by 30 weeks of age, with manifestation in only 20% mice. This was associated with reduced insulitis and islet infiltration of CD4+and CD8+cells as shown in FIGS. 13 and 14 using the histopathological analysis according to Salomon et al., 2000, Immunity 12:431-440. In contrast, 90% of mice developed diabetes by 17-20 week of age in untreated groups. No animals receiving DC infusion became diabetic within the first 20 weeks post-treatment. In contrast, all of the untreated or mock-treated NOD mice developed diabetes by 17-20 weeks of age, as shown in FIG. 15.

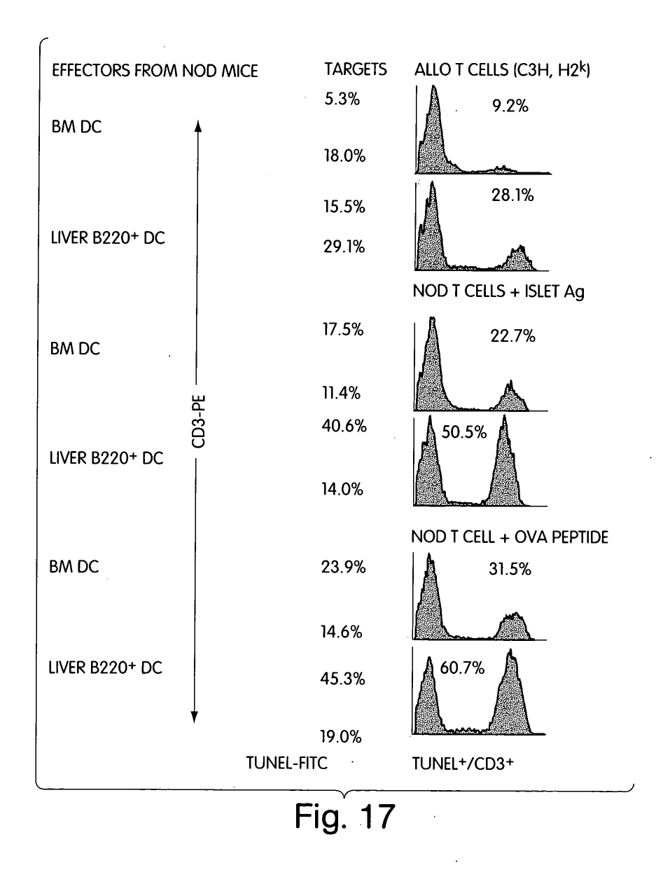

Liver-derived DEC205⁺B22⁰+CD19⁻cells prevent autoimmune responses in NOD mice via deletion of activated T cells. The antigen presenting capacity of liver-derived DEC205⁺B22⁰+CD19⁻cells was manifested as a significant increase in OVA-specific T cells identified by specific mAb KJI.26 in draining lymph nodes (LN) of the mice primed with OVA pulsed-DC at day 2-3 following immunization. These antigen-specific T cells (KJ1.26+T cells) rapidly declined thereafter in mice that were primed with OVA-pulsed liver-derived DEC205⁺B22⁰+CD19⁻cells. However, KJ1.26+T cells continued to proliferate up to 7 days after immunization in BM DC primed groups, indicating deletion of these T cells following activation (FIG. 16). The activated T cell apoptosis was determined by flow cytometric analysis with anti -CD3 and TUNEL double staining. As shown in FIG. 17, compared with BM DC, islet Ag or OVA peptide-pulsed-liver-derived DEC205⁺B22₀+CD19⁻cells propagated from NOD mice induced high levels of apoptosis in allogenic (C3H, H2 k) T cells or T cells of NOD mice.

To determine whether the T cell apoptosis is due to an inappropriate activation or direct killing by DC, 3[H]TdR labeled Con A activated or allo-antigen activated T cells from NOD mice were co-cultured with liver-derived DEC205⁺B22⁰+CD19⁻cells or BM DC at 4/1 ratio of T/DC for 8 hr. The direct killing effect of DC on T cell was confirmed by DNA fragmentation assay. The liver-derived DEC205⁺B22⁰+CD19⁻cells induced significantly higher levels of apoptosis in activated T cells than that of BM DC. The data of this study demonstrated that tolerogenic liver DC effectively prevented diabetes development in NOD mice, which likely transpires via promoting death of antigen-specific T cells immediately after activation

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the instant invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed:

1. A tolerogenic dendritic cell (DC) having surface antigens DEC205 and B220, but not CD19.

2. The dendritic cell of claim 1, which is mammalian.

3. The dendritic cell of claim 1, which is human.

4. The dendritic cell of claim 3, which has been obtained from liver or bone marrow.

5. A tolerogenic dendritic cell of claim 1, which has been engineered to have increased expression of an immunosuppressive protein.

6. A tolerogenic dendritic cell of claim 5, wherein the immunosuppressive protein is selected from the group consisting of: interleukin 4, interleukin 6, interleukin 10, transforming growth factor β, interferon γ, macrophage migration inhibitory factor and lymphotoxin β (LTB).

7. A tolerogenic dendritic cell of claim 1, which has been engineered to have decreased expression of an immunostimulatory protein.

8. A tolerogenic dendritic cell of claim 7, wherein the immunostimulatory protein is selected from the group consisting of: interleukin 12 and tumor necrosis factor α.

9. A pharmaceutical preparation comprised of substantially enriched tolerogenic dendritic cells having surface antigens DEC205 and B220, but not CD19.

10. The pharmaceutical preparation of claim 9, wherein the dendritic cell is mammalian.

11. The pharmaceutical preparation of claim 9, wherein the dendritic cell is human.

12. The pharmaceutical preparation of claim 9, wherein the dendritic cell is obtained from liver or bone marrow.

13. A pharmaceutical preparation of claim 9, which additionally comprises an immunosuppressive agent.

14. A pharmaceutical preparation of claim 13, wherein the immunosuppressive agent is selected from the group consisting of: a small molecule, protein, nucleic acid, lipid and carbohydrate,

15. A pharmaceutical preparation of claim 14, wherein the immunosuppressive agent is a small molecule selected from the group consisting of: azathioprine, tacrolimus, cyclosporin, cyclophosphamide, daclizumab, mycophenolate mofetil, prednisone, and sirolimus.

16. A method of obtaining a substantially enriched population of tolerogenic dendritic cells, comprising:
(a) harvesting nonparenchymal cells from the tissue of a subject,
b) culturing the nonparenchymal cell population with interleukin3 (IL-3) and an activator of CD40 ligand, and
(c) substantially enriching the culture of step (b) for tolerogenic dendritic cells.

17. A method of claim 16, wherein the donor tissue is bone marrow or liver.

18. A method of claim 16, wherein prior to step (b), the nonparenchymal cells are depleted of T cells, B cells, NK cells, granular cells and macrophages.

19. A method of claim 16, wherein step (c) is performed using metrizamide gradient centrifugation.

20. A method of claim 16, wherein step (c) is performed using fluorescence activated cell sorting.

21. A method of claim 20, which employs fluorescently labeled antibodies selected from the group consisting of: anti-DEC205, anti-B220 and anti-CD19.

22. A method of claim 16, wherein the tissue is human.

23. A method of enhancing tolerogenicity in a subject comprising administering an effective amount of the pharmaceutical composition of claim 9.

24. A method of enhancing tolerogenicity in a subject comprising administering an effective amount of the pharmaceutical composition of claim 13.

25. A method of claim 23, wherein the administration is intravenous.

26. A method of claim 23, wherein the subject has received a transplant.

27. A method of claim 23, wherein the method is performed prior to performance of the transplant.

28. A method of claim 23, wherein the method is performed in conjunction with the transplant.

29. A method of claim 23, wherein the method is performed within at least two weeks after the transplant.

30. A method of claim 21, wherein the subject has or is susceptible to developing an autoimmune disease.

31. A method of claim 30, wherein the autoimmune disease is type 1 diabetes.
